SEQUENCE LISTING (35 sequences)

SEQ ID NO: 1 - DNA, 8574 bp, Artificial Sequence
  Plasmid
aaactcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcagggccccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgatcgggccccctgcaggataaaaaaattgtagataaattttataaaatagttttatctacaatttttttatcaggaaacagctatgaccgcggccgctgtatccatatgaccatgattacgaattccccggatcgagatagtatatgatgcatattctttaaatatagataaagttatagaagcaatagaagatttaggatttactgtaatataaattacacttttaaaaagtttaaaaacatgatacaataagttatggttggaattgttatccgctcacaattccaacttatgattaaaattttaaggaggtgtatttcataatgtgtcgaataacgctttacaaacaattattaacgcccggttaccaggcgaagaggggctgtggcagattcatctgcaggacggaaaaatcagcgccattgatgcgcaatccggcgtgatgcccataactgaaaacagcctggatgccgaacaaggtttagttataccgccgtttgtggagccacatattcacctggacaccacgcaaaccgccggacaaccgaactggaatcagtccggcacgctgtttgaaggcattgaacgctgggccgagcgcaaagcgttattaacccatgacgatgtgaaacaacgcgcatggcaaacgctgaaatggcagattgccaacggcattcagcatgtgcgtacccatgtcgatgtttcggatgcaacgctaactgcgctgaaagcaatgctggaagtgaagcaggaagtcgcgccgtggattgatctgcaaatcgtcgccttccctcaggaagggattttgtcgtatcccaacggtgaagcgttgctggaagaggcgttacgcttaggggcagatgtagtgggggcgattccgcattttgaatttacccgtgaatacggcgtggagtcgctgcataaaaccttcgccctggcgcaaaaatacgaccgtctcatcgacgttcactgtgatgagatcgatgacgagcagtcgcgctttgtcgaaaccgttgctgccctggcgcaccatgaaggcatgggcgcgcgagtcaccgccagccacaccacggcaatgcactcctataacggggcgtatacctcacgcctgttccgcttgctgaaaatgtccggtattaactttgtcgccaacccgctggtcaatattcatctgcaaggacgtttcgatacgtatccaaaacgtcgcggcatcacgcgcgttaaagagatgctggagtccggcattaacgtctgctttggtcacgatgatgtcttcgatccgtggtatccgctgggaacggcgaatatgctgcaagtgctgcatatggggctgcatgtttgccagttgatgggctacgggcagattaacgatggcctgaatttaatcacccaccacagcgcaaggacgttgaatttgcaggattacggcattgccgccggaaacagcgccaacctgattatcctgccggctgaaaatgggtttgatgcgctgcgccgtcaggttccggtacgttattcggtacgtggcggcaaggtgattgccagcacacaaccggcacaaaccaccgtatatctggagcagccagaagccatcgattacaaacgttgaacgactgggttacagcgagcttagtttaaagggcgaattcgagctcggtacccggggatcctctagagtcgacgtcacgcgtccatggagatctcgaggcctgcagacatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctagcataaaaataagaagcctgcatttgcaggcttcttatttttatggcgcgccgttctgaatccttagctaatggttcaacaggtaactatgacgaagatagcaccctggataagtctgtaatggattctaaggcatttaatgaagacgtgtatataaaatgtgctaatgaaaaagaaaatgcgttaaaagagcctaaaatgagttcaaatggttttgaaattgattggtagtttaatttaatatattttttctattggctatctcgatacctatagaatcttctgttcacttttgtttttgaaatataaaaaggggctttttagccccttttttttaaaactccggaggagtttcttcattcttgatactatacgtaactattttcgatttgacttcattgtcaattaagctagtaaaatcaatggttaaaaaacaaaaaacttgcatttttctacctagtaatttataattttaagtgtcgagtttaaaagtataatttaccaggaaaggagcaagttttttaataaggaaaaatttttccttttaaaattctatttcgttatatgactaattataatcaaaaaaatgaaaataaacaagaggtaaaaactgctttagagaaatgtactgataaaaaaagaaaaaatcctagatttacgtcatacatagcacctttaactactaagaaaaatattgaaaggacttccacttgtggagattatttgtttatgttgagtgatgcagacttagaacattttaaattacataaaggtaatttttgcggtaatagattttgtccaatgtgtagttggcgacttgcttgtaaggatagtttagaaatatctattcttatggagcatttaagaaaagaagaaaataaagagtttatatttttaactcttacaactccaaatgtaaaaagttatgatcttaattattctattaaacaatataataaatcttttaaaaaattaatggagcgtaaggaagttaaggatataactaaaggttatataagaaaattagaagtaacttaccaaaaggaaaaatacataacaaaggatttatggaaaataaaaaaagattattatcaaaaaaaaggacttgaaattggtgatttagaacctaattttgatacttataatcctcattttcatgtagttattgcagttaataaaagttattttacagataaaaattattatataaatcgagaaagatggttggaattatggaagtttgctactaaggatgattctataactcaagttgatgttagaaaagcaaaaattaatgattataaagaggtttacgaacttgcgaaatattcagctaaagacactgattatttaatatcgaggccagtatttgaaattttttataaagcattaaaaggcaagcaggtattagtttttagtggattttttaaagatgcacacaaattgtacaagcaaggaaaacttgatgtttataaaaagaaagatgaaattaaatatgtctatatagtttattataattggtgcaaaaaacaatatgaaaaaactagaataagggaacttacggaagatgaaaaagaagaattaaatcaagatttaatagatgaaatagaaatagattaaagtgtaactatactttatatatatatgattaaaaaaataaaaaacaacagcctattaggttgttgttttttattttctttattaatttttttaatttttagtttttagttcttttttaaaataagtttcagcctctttttcaatattttttaaagaaggagtatttgcatgaattgccttttttctaacagacttaggaaatattttaacagtatcttcttgcgccggtgattttggaacttcataacttactaatttataattattattttcttttttaattgtaacagttgcaaaagaagctgaacctgttccttcaactagtttatcatcttcaatataatattcttgacctatatagtataaatatatttttattatatttttacttttttctgaatctattattttataatcataaaaagttttaccaccaaaagaaggttgtactccttctggtccaacatatttttttactatattatctaaataatttttgggaactggtgttgtaatttgattaatcgaacaaccagttatacttaaaggaattataactataaaaatatataggattatctttttaaatttcattattggcctcctttttattaaatttatgttaccataaaaaggacataacgggaatatgtagaatatttttaatgtagacaaaattttacataaatataaagaaaggaagtgtttgtttaaattttatagcaaactatcaaaaattagggggataaaaatttatgaaaaaaaggttttcgatgttatttttatgtttaactttaatagtttgtggtttatttacaaattcggccggccgaagcaaacttaagagtgtgttgatagtgcagtatcttaaaattttgtataataggaattgaagttaaattagatgctaaaaatttgtaattaagaaggagtgattacatgaacaaaaatataaaatattctcaaaactttttaacgagtgaaaaagtctggcgaaagggggatgtgctgcaaggcgattaagttgggtaacgccagggttttcccagtcacgacgttgtaaaacgacggccagtgaattcgccctttcagtttacaacttgtaaagacacatactatatccatagtataataaaattattatattgatatagtaagcaaataataaagaaaatttttatatttattaaaatagcgaaacggtataaccctagtggttataccgtttcgctgcttgtaatacgcatttatagccaatgccttaattaaaaagccttactttttatgtcttgtttatttaaccatactatgttctagtcttaatttatcagcaatcattgctataaattctgaattagttggttttcctttagtattgtgtaccgtatatccaaataattgatttattgtgtcaacttttcctctactccatgcaacctctattgcatgtcttattgctctttcaactctacttggtgttgtattgaattttttagctatacttggatataactcttttgttactgcacctaaaagctcaacattatcaataaccatttttattgcttctcttaaatataaatatccttttatatgagctggtactcctatttcatgtattatatttgtgatttctgtctctatatttcctacattcttaacaaaatcacttctagtcatttgagtctcttgaactggtctaggttttggctcaacttgtgtaactctattagaaactagctctctaattctgtttactgaccggtctaaagaggtccctagcgcctacggggaatttgtatcgataaggggtacaaattcccactaagcgctcggcggggatcgatcccgggtacgtacccggcagtttttctttttcggcaagtgttcaagaagttattaagtcgggagtgcagtcgaagtgggcaagttgaaaaattcacaaaaatgtggtataatatctttgttcattagagcgataaacttgaatttgagagggaacttagatggtatttgaaaaaattgataaaaatagttggaacagaaaagagtattttgaccactactttgcaagtgtaccttgtacctacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaacttcaggtttgtctgtaactaaaaacaagtatttaagcaaaaacatcgtagaaatacggtgttttttgttaccctaaaatctacaattttatacataaccacaggttagtacaaagaccttgtgtttctttttgaaaggcttaaaacaaggatttttccttgatttaagccccgaaaagcaacacaaccaaggttttagtatcaatctgtggtttttatattttcagagaccggtcagtaaacacaacaaaatcaaatggctttactatgtagtaatctgctcctagatttattgcgctttgagttatcttatcttgacctactgctgatagtactattatttttggcattttaggtatatccatagtattcaatttttcaattacacctaatccatctagatgtggcattattacatctagtattaataagtccggttgtgtcttttttactaagtctaatgcttcaattccatccttagctatgcctaatatatcgatatcatcttcattagacaaatactcttttaatacctgacaaaaatccttattgtcatctgctaaaactattttgattttttccactaaaaatccccccattaaaaaacatcttcttattacagaaaactaaactttttacacaattgcttcctaattatctactctaaagcatattcgacactccaaaattatattcctcctagataattgataaaataataaaattttttaatgcctcacctacaattttatcatttcatctataaaaataccaagggcgaattcgag

SEQ ID NO: 2 - DNA, 6778 bp, Artificial Sequence
  Plasmid
caggataaaaaaattgtagataaattttataaaatagttttatctacaatttttttatcaggaaacagctatgaccgcggccgctgtatccatatgaccatgattacgaattccccggatcgagatagtatatgatgcatattctttaaatatagataaagttatagaagcaatagaagatttaggatttactgtaatataaattacacttttaaaaagtttaaaaacatgatacaataagttatggttggaattgttatccgctcacaattccaacttatgattaaaattttaaggaggtgtatttcataatgtgtcgaataacgctttacaaacaattattaacgcccggttaccaggcgaagaggggctgtggcagattcatctgcaggacggaaaaatcagcgccattgatgcgcaatccggcgtgatgcccataactgaaaacagcctggatgccgaacaaggtttagttataccgccgtttgtggagccacatattcacctggacaccacgcaaaccgccggacaaccgaactggaatcagtccggcacgctgtttgaaggcattgaacgctgggccgagcgcaaagcgttattaacccatgacgatgtgaaacaacgcgcatggcaaacgctgaaatggcagattgccaacggcattcagcatgtgcgtacccatgtcgatgtttcggatgcaacgctaactgcgctgaaagcaatgctggaagtgaagcaggaagtcgcgccgtggattgatctgcaaatcgtcgccttccctcaggaagggattttgtcgtatcccaacggtgaagcgttgctggaagaggcgttacgcttaggggcagatgtagtgggggcgattccgcattttgaatttacccgtgaatacggcgtggagtcgctgcataaaaccttcgccctggcgcaaaaatacgaccgtctcatcgacgttcactgtgatgagatcgatgacgagcagtcgcgctttgtcgaaaccgttgctgccctggcgcaccatgaaggcatgggcgcgcgagtcaccgccagccacaccacggcaatgcactcctataacggggcgtatacctcacgcctgttccgcttgctgaaaatgtccggtattaactttgtcgccaacccgctggtcaatattcatctgcaaggacgtttcgatacgtatccaaaacgtcgcggcatcacgcgcgttaaagagatgctggagtccggcattaacgtctgctttggtcacgatgatgtcttcgatccgtggtatccgctgggaacggcgaatatgctgcaagtgctgcatatggggctgcatgtttgccagttgatgggctacgggcagattaacgatggcctgaatttaatcacccaccacagcgcaaggacgttgaatttgcaggattacggcattgccgccggaaacagcgccaacctgattatcctgccggctgaaaatgggtttgatgcgctgcgccgtcaggttccggtacgttattcggtacgtggcggcaaggtgattgccagcacacaaccggcacaaaccaccgtatatctggagcagccagaagccatcgattacaaacgttgaacgactgggttacagcgagcttagtttaaagggcgaattcgagctcggtacccggggatcctctagagtcgacgtcacgcgtccatggagatctcgaggcctgcagacatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctagcataaaaataagaagcctgcatttgcaggcttcttatttttatggcgcgccgttctgaatccttagctaatggttcaacaggtaactatgacgaagatagcaccctggataagtctgtaatggattctaaggcatttaatgaagacgtgtatataaaatgtgctaatgaaaaagaaaatgcgttaaaagagcctaaaatgagttcaaatggttttgaaattgattggtagtttaatttaatatattttttctattggctatctcgatacctatagaatcttctgttcacttttgtttttgaaatataaaaaggggctttttagccccttttttttaaaactccggaggagtttcttcattcttgatactatacgtaactattttcgatttgacttcattgtcaattaagctagtaaaatcaatggttaaaaaacaaaaaacttgcatttttctacctagtaatttataattttaagtgtcgagtttaaaagtataatttaccaggaaaggagcaagttttttaataaggaaaaatttttccttttaaaattctatttcgttatatgactaattataatcaaaaaaatgaaaataaacaagaggtaaaaactgctttagagaaatgtactgataaaaaaagaaaaaatcctagatttacgtcatacatagcacctttaactactaagaaaaatattgaaaggacttccacttgtggagattatttgtttatgttgagtgatgcagacttagaacattttaaattacataaaggtaatttttgcggtaatagattttgtccaatgtgtagttggcgacttgcttgtaaggatagtttagaaatatctattcttatggagcatttaagaaaagaagaaaataaagagtttatatttttaactcttacaactccaaatgtaaaaagttatgatcttaattattctattaaacaatataataaatcttttaaaaaattaatggagcgtaaggaagttaaggatataactaaaggttatataagaaaattagaagtaacttaccaaaaggaaaaatacataacaaaggatttatggaaaataaaaaaagattattatcaaaaaaaaggacttgaaattggtgatttagaacctaattttgatacttataatcctcattttcatgtagttattgcagttaataaaagttattttacagataaaaattattatataaatcgagaaagatggttggaattatggaagtttgctactaaggatgattctataactcaagttgatgttagaaaagcaaaaattaatgattataaagaggtttacgaacttgcgaaatattcagctaaagacactgattatttaatatcgaggccagtatttgaaattttttataaagcattaaaaggcaagcaggtattagtttttagtggattttttaaagatgcacacaaattgtacaagcaaggaaaacttgatgtttataaaaagaaagatgaaattaaatatgtctatatagtttattataattggtgcaaaaaacaatatgaaaaaactagaataagggaacttacggaagatgaaaaagaagaattaaatcaagatttaatagatgaaatagaaatagattaaagtgtaactatactttatatatatatgattaaaaaaataaaaaacaacagcctattaggttgttgttttttattttctttattaatttttttaatttttagtttttagttcttttttaaaataagtttcagcctctttttcaatattttttaaagaaggagtatttgcatgaattgccttttttctaacagacttaggaaatattttaacagtatcttcttgcgccggtgattttggaacttcataacttactaatttataattattattttcttttttaattgtaacagttgcaaaagaagctgaacctgttccttcaactagtttatcatcttcaatataatattcttgacctatatagtataaatatatttttattatatttttacttttttctgaatctattattttataatcataaaaagttttaccaccaaaagaaggttgtactccttctggtccaacatatttttttactatattatctaaataatttttgggaactggtgttgtaatttgattaatcgaacaaccagttatacttaaaggaattataactataaaaatatataggattatctttttaaatttcattattggcctcctttttattaaatttatgttaccataaaaaggacataacgggaatatgtagaatatttttaatgtagacaaaattttacataaatataaagaaaggaagtgtttgtttaaattttatagcaaactatcaaaaattagggggataaaaatttatgaaaaaaaggttttcgatgttatttttatgtttaactttaatagtttgtggtttatttacaaattcggccggccagtgggcaagttgaaaaattcacaaaaatgtggtataatatctttgttcattagagcgataaacttgaatttgagagggaacttagatggtatttgaaaaaattgataaaaatagttggaacagaaaagagtattttgaccactactttgcaagtgtaccttgtacctacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaacttcaggtttgtctgtaactaaaaacaagtatttaagcaaaaacatcgtagaaatacggtgttttttgttaccctaagtttaaactcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcagggccccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgatcgggccccctg

SEQ ID NO: 3 - DNA, 6000 bp, Artificial Sequence
  Plasmid
ataaaaaaattgtagataaattttataaaatagttttatctacaatttttttatcaggaaacagctatgaccgcggccgctgtatccatatgaccatgattacgaattccccggatcgagatagtatatgatgcatattctttaaatatagataaagttatagaagcaatagaagatttaggatttactgtaatataaattacacttttaaaaagtttaaaaacatgatacaataagttatggttggaattgttatccgctcacaattccaacttatgattaaaattttaaggaggtgtatttcataatgtgtcgaataacgctttacaaacaattattaacgcccggttaccaggcgaagaggggctgtggcagattcatctgcaggacggaaaaatcagcgccattgatgcgcaatccggcgtgatgcccataactgaaaacagcctggatgccgaacaaggtttagttataccgccgtttgtggagccacatattcacctggacaccacgcaaaccgccggacaaccgaactggaatcagtccggcacgctgtttgaaggcattgaacgctgggccgagcgcaaagcgttattaacccatgacgatgtgaaacaacgcgcatggcaaacgctgaaatggcagattgccaacggcattcagcatgtgcgtacccatgtcgatgtttcggatgcaacgctaactgcgctgaaagcaatgctggaagtgaagcaggaagtcgcgccgtggattgatctgcaaatcgtcgccttccctcaggaagggattttgtcgtatcccaacggtgaagcgttgctggaagaggcgttacgcttaggggcagatgtagtgggggcgattccgcattttgaatttacccgtgaatacggcgtggagtcgctgcataaaaccttcgccctggcgcaaaaatacgaccgtctcatcgacgttcactgtgatgagatcgatgacgagcagtcgcgctttgtcgaaaccgttgctgccctggcgcaccatgaaggcatgggcgcgcgagtcaccgccagccacaccacggcaatgcactcctataacggggcgtatacctcacgcctgttccgcttgctgaaaatgtccggtattaactttgtcgccaacccgctggtcaatattcatctgcaaggacgtttcgatacgtatccaaaacgtcgcggcatcacgcgcgttaaagagatgctggagtccggcattaacgtctgctttggtcacgatgatgtcttcgatccgtggtatccgctgggaacggcgaatatgctgcaagtgctgcatatggggctgcatgtttgccagttgatgggctacgggcagattaacgatggcctgaatttaatcacccaccacagcgcaaggacgttgaatttgcaggattacggcattgccgccggaaacagcgccaacctgattatcctgccggctgaaaatgggtttgatgcgctgcgccgtcaggttccggtacgttattcggtacgtggcggcaaggtgattgccagcacacaaccggcacaaaccaccgtatatctggagcagccagaagccatcgattacaaacgttgaacgactgggttacagcgagcttagtttaaagggcgaattcgagctcggtacccggggatcctctagagtcgacgtcacgcgtccatggagatctcgaggcctgcagacatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctagcataaaaataagaagcctgcatttgcaggcttcttatttttatggcgcgccgccattatttttttgaacaattgacaattcatttcttattttttattaagtgatagtcaaaaggcataacagtgctgaatagaaagaaatttacagaaaagaaaattatagaatttagtatgattaattatactcatttatgaatgtttaattgaatacaaaaaaaaatacttgttatgtattcaattacgggttaaaatatagacaagttgaaaaatttaataaaaaaataagtcctcagctcttatatattaagctaccaacttagtatataagccaaaacttaaatgtgctaccaacacatcaagccgttagagaactctatctatagcaatatttcaaatgtaccgacatacaagagaaacattaactatatatattcaatttatgagattatcttaacagatataaatgtaaattgcaataagtaagatttagaagtttatagcctttgtgtattggaagcagtacgcaaaggcttttttatttgataaaaattagaagtatatttattttttcataattaatttatgaaaatgaaagggggtgagcaaagtgacagaggaaagcagtatcttatcaaataacaaggtattagcaatatcattattgactttagcagtaaacattatgacttttatagtgcttgtagctaagtagtacgaaagggggagctttaaaaagctccttggaatacatagaattcataaattaatttatgaaaagaagggcgtatatgaaaacttgtaaaaattgcaaagagtttattaaagatactgaaatatgcaaaatacattcgttgatgattcatgataaaacagtagcaacctattgcagtaaatacaatgagtcaagatgtttacataaagggaaagtccaatgtattaattgttcaaagatgaaccgatatggatggtgtgccataaaaatgagatgttttacagaggaagaacagaaaaaagaacgtacatgcattaaatattatgcaaggagctttaaaaaagctcatgtaaagaagagtaaaaagaaaaaataatttatttattaatttaatattgagagtgccgacacagtatgcactaaaaaatatatctgtggtgtagtgagccgatacaaaaggatagtcactcgcattttcataatacatcttatgttatgattatgtgtcggtgggacttcacgacgaaaacccacaataaaaaaagagttcggggtagggttaagcatagttgaggcaactaaacaatcaagctaggatatgcagtagcagaccgtaaggtcgttgtttaggtgtgttgtaatacatacgctattaagatgtaaaaatacggataccaatgaagggaaaagtataatttttggatgtagtttgtttgttcatctatgggcaaactacgtccaaagccgtttccaaatctgctaaaaagtatatcctttctaaaatcaaagtcaagtatgaaatcataaataaagtttaattttgaagttattatgatattatgtttttctattaaaataaattaagtatatagaatagtttaataatagtatatacttaatgtgataagtgtctgacagtgtcacagaaaggatgattgttatggattataagcggccggccagtgggcaagttgaaaaattcacaaaaatgtggtataatatctttgttcattagagcgataaacttgaatttgagagggaacttagatggtatttgaaaaaattgataaaaatagttggaacagaaaagagtattttgaccactactttgcaagtgtaccttgtacctacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaacttcaggtttgtctgtaactaaaaacaagtatttaagcaaaaacatcgtagaaatacggtgttttttgttaccctaagtttaaactcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcagggccccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgatcgggccccctgcagg

SEQ ID NO: 4 - DNA, 7821 bp, Artificial Sequence
  Plasmid
ataaaaaaattgtagataaattttataaaatagttttatctacaatttttttatcaggaaacagctatgaccgcggccgctgtatccatatgaccatgattacgaattccccggatcgagatagtatatgatgcatattctttaaatatagataaagttatagaagcaatagaagatttaggatttactgtaatataaattacacttttaaaaagtttaaaaacatgatacaataagttatggttggaattgttatccgctcacaattccaacttatgattaaaattttaaggaggtgtatttcataatgtgtcgaataacgctttacaaacaattattaacgcccggttaccaggcgaagaggggctgtggcagattcatctgcaggacggaaaaatcagcgccattgatgcgcaatccggcgtgatgcccataactgaaaacagcctggatgccgaacaaggtttagttataccgccgtttgtggagccacatattcacctggacaccacgcaaaccgccggacaaccgaactggaatcagtccggcacgctgtttgaaggcattgaacgctgggccgagcgcaaagcgttattaacccatgacgatgtgaaacaacgcgcatggcaaacgctgaaatggcagattgccaacggcattcagcatgtgcgtacccatgtcgatgtttcggatgcaacgctaactgcgctgaaagcaatgctggaagtgaagcaggaagtcgcgccgtggattgatctgcaaatcgtcgccttccctcaggaagggattttgtcgtatcccaacggtgaagcgttgctggaagaggcgttacgcttaggggcagatgtagtgggggcgattccgcattttgaatttacccgtgaatacggcgtggagtcgctgcataaaaccttcgccctggcgcaaaaatacgaccgtctcatcgacgttcactgtgatgagatcgatgacgagcagtcgcgctttgtcgaaaccgttgctgccctggcgcaccatgaaggcatgggcgcgcgagtcaccgccagccacaccacggcaatgcactcctataacggggcgtatacctcacgcctgttccgcttgctgaaaatgtccggtattaactttgtcgccaacccgctggtcaatattcatctgcaaggacgtttcgatacgtatccaaaacgtcgcggcatcacgcgcgttaaagagatgctggagtccggcattaacgtctgctttggtcacgatgatgtcttcgatccgtggtatccgctgggaacggcgaatatgctgcaagtgctgcatatggggctgcatgtttgccagttgatgggctacgggcagattaacgatggcctgaatttaatcacccaccacagcgcaaggacgttgaatttgcaggattacggcattgccgccggaaacagcgccaacctgattatcctgccggctgaaaatgggtttgatgcgctgcgccgtcaggttccggtacgttattcggtacgtggcggcaaggtgattgccagcacacaaccggcacaaaccaccgtatatctggagcagccagaagccatcgattacaaacgttgaacgactgggttacagcgagcttagtttaaagggcgaattcgagctcggtacccggggatcctctagagtcgacgtcacgcgtccatggagatctcgaggcctgcagacatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctagcataaaaataagaagcctgcatttgcaggcttcttatttttatggcgcgcccgcccttaagtctaaaaattaggggagatgtaaggatttgggaaaaatagaagatgttataatcataaatatggtattcgtaggcttaaagtcaaaaaggaggtgaaatataaatagatttttagctaaattaagtaagaaataggaggagatttattgaacaaaaaattagaaaaaccatttgtatataagagagagtacgatttgactggatatgatgttgaaattttacaaaaatatgagttagaacaagcaatatatgtttatgttgggagtagttgtgcatataacatgagagctagaagtagtaaatggagataccatataagaacaaataataagtctatatgttgtaacattaaaaattttatacataacttggaattgttttataaaatggaattaaagttgtcagataatattattaatgataagctatactatagcaatatagcagagtttgaagaatttgaaacactagaaaaagctagagaggtagaaagtactattataagtcaatatcaatttttagattctataaatcacatgttaaaacaaaaaataattttattgagtaataaggatagtgtgttaaacataactaaaaatggaaatacaaattatttgaaagtaaaaaataaatacatagaaaaacataagaacaagccaataatgagataccatatcaactgtcaattcaatacagatggaagtgtcaaaagtattacacaggagtttgaaccaatattggaattaaacaaaaaaaataccctaagccgaccaagcagagtatttttaaaataatattttaagataacaacaaaatgagataatactactagacaatgacaactcaactaccaattgagtttatggagctaccaactccaatatcggtctaactgattaagtatctgtagttatataataatattgctatcaattttagcatcttaacaatattattatacatactaagctaaaattattcaatagttgtaaaagttgattagtcaataagtatatatttaatgtagtgttatctcttaaaaaaactagataaggagataataaatatatggaacaattagattcaaaatataagttgaaaaaatttctaatggcagtatttagagatggtataggacaaggaaataatcttattgataatgaatatgttagagtatttcaaaataataaaagtaatagtaaacaattagaactcggagaagaatttaaagaatatagtaaaacaactttttttaaaaatatagatgatatagtagaatttaccttcgcaaaaaatatttattatgaaaatacattttttaacctatgtactactgatggaaaagcaggaaccaatgaaaacttaataaatagatatgcattaggatttgattttgacaaaaaagaattaggacaaggttttaattataaagatataattaatttatttactaagataggattacattatcatatcctagttgatagtggaaatggattccatgtttatgtgctaattaataaaactaataacattaagttagtatcagaagttacaaatacattaataaataaattgggtgcagataaacaagcaaatttatctactcaagtattaagagtaccttatacatataatattaaaaatactactaaacaagtaaaaataatacaccaagacaaaaatatatatagatatgacatagaaaagttagctaaaaaatattgcaaagatgtaaaaacagtaggtaatactaatacaaaatatatattagatagtaagctaccaaattgtatagtagatattttaaaaaatggtagtaaagatggacataaaaacctagatttgcaaaaaatagttgtgactttaagattgaggaataaaagtttaagtcaagtaatatccgttgctagagaatggaactatatatcacaaaatagtctttcaaatagtgagctagaatatcaagtcaagtatatgtatgagaaacttaaaacggttaattttggttgtactggttgtgagtttaatagtgattgttggaataaaatagaatcagattttatatatagtgatgaagatactttgttcaatatgccacataagcactcaaaggatttgaaatataagaataggaaaggggttaaaataatgactggtaatcaattgtttatctataatgtgttacttaacaataaagatagagaattaaacatagacgatataatggagctgataacctataaacgtaagaagaaagttaaaaacattgttatgagtgaaaagacattaagagaaacattaaaagaacttcaacataatgattatattacaaaaacaaaaggtgttacaaagctaggaataaaagatacatacaatgtaaaagaagttagatgtaatatagataaacaatatactattagttactttgttaccatggcagtaatttggggaataatttcaactgaagaattaagattatatactcacatgagatataagcaagatttattggtcaaagatgataaaataaaaggaaatatattaagaattaatcaagaggaattagcaaaagatttaggagtaacacagcaaagaatttcaaatatgatagaatctttattagatactaaaattttagatgtatgggaaactaaaataaatgatagaggatttatgtactatacatatagattaaacaagtagatttttgataggattagaattgattttctagtcctatttttatgcaaaaaaactaattaataaaaatttcttttggtaaaataattgtacgagaattgcaaaaaaaaaatggcatcaaagtattgaaattaagccgttttaaaaatttcttttggtaaaataattctacatatatatgtagtatatatatatatgttttttagagaatgtataactagaatatagagctagaatatagagaatgtataactagaatatagagctagaatatagagaatgtataactagaatatagagctagaatatagagaatgtataactagaatatagagctagaatatagagaatgtataactagaatatagagctagaatatagagaatgtataactagaatatagagctagaatatagagaatgtataactagaatatagagctagaatcctaattagtaggtgcttttttaaaacaagttaaaaatcaaaaatagtattagtaagcattggaaatgctagattctaaaatagaaaagtaaaaaattggtgcactatctaaacttatctatatcgctttttccgtcgtttggttctctagttacgatacaggggatatgcttatattgagttatagtactaatcagtgcttaatatagttaataaaattatagttaccatagtttagtaactatgatgtatgttagttagaaacttgcatttcggccggccagtgggcaagttgaaaaattcacaaaaatgtggtataatatctttgttcattagagcgataaacttgaatttgagagggaacttagatggtatttgaaaaaattgataaaaatagttggaacagaaaagagtattttgaccactactttgcaagtgtaccttgtacctacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaacttcaggtttgtctgtaactaaaaacaagtatttaagcaaaaacatcgtagaaatacggtgttttttgttaccctaagtttaaactcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcagggccccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgatcgggccccctgcagg

SEQ ID NO: 5 - DNA, 5253 bp, Artificial Sequence
  Plasmid
ataaaaaaattgtagataaattttataaaatagttttatctacaatttttttatcaggaaacagctatgaccgcggccgctgtatccatatgaccatgattacgaattccccggatcgagatagtatatgatgcatattctttaaatatagataaagttatagaagcaatagaagatttaggatttactgtaatataaattacacttttaaaaagtttaaaaacatgatacaataagttatggttggaattgttatccgctcacaattccaacttatgattaaaattttaaggaggtgtatttcataatgtgtcgaataacgctttacaaacaattattaacgcccggttaccaggcgaagaggggctgtggcagattcatctgcaggacggaaaaatcagcgccattgatgcgcaatccggcgtgatgcccataactgaaaacagcctggatgccgaacaaggtttagttataccgccgtttgtggagccacatattcacctggacaccacgcaaaccgccggacaaccgaactggaatcagtccggcacgctgtttgaaggcattgaacgctgggccgagcgcaaagcgttattaacccatgacgatgtgaaacaacgcgcatggcaaacgctgaaatggcagattgccaacggcattcagcatgtgcgtacccatgtcgatgtttcggatgcaacgctaactgcgctgaaagcaatgctggaagtgaagcaggaagtcgcgccgtggattgatctgcaaatcgtcgccttccctcaggaagggattttgtcgtatcccaacggtgaagcgttgctggaagaggcgttacgcttaggggcagatgtagtgggggcgattccgcattttgaatttacccgtgaatacggcgtggagtcgctgcataaaaccttcgccctggcgcaaaaatacgaccgtctcatcgacgttcactgtgatgagatcgatgacgagcagtcgcgctttgtcgaaaccgttgctgccctggcgcaccatgaaggcatgggcgcgcgagtcaccgccagccacaccacggcaatgcactcctataacggggcgtatacctcacgcctgttccgcttgctgaaaatgtccggtattaactttgtcgccaacccgctggtcaatattcatctgcaaggacgtttcgatacgtatccaaaacgtcgcggcatcacgcgcgttaaagagatgctggagtccggcattaacgtctgctttggtcacgatgatgtcttcgatccgtggtatccgctgggaacggcgaatatgctgcaagtgctgcatatggggctgcatgtttgccagttgatgggctacgggcagattaacgatggcctgaatttaatcacccaccacagcgcaaggacgttgaatttgcaggattacggcattgccgccggaaacagcgccaacctgattatcctgccggctgaaaatgggtttgatgcgctgcgccgtcaggttccggtacgttattcggtacgtggcggcaaggtgattgccagcacacaaccggcacaaaccaccgtatatctggagcagccagaagccatcgattacaaacgttgaacgactgggttacagcgagcttagtttaaagggcgaattcgagctcggtacccggggatcctctagagtcgacgtcacgcgtccatggagatctcgaggcctgcagacatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctagcataaaaataagaagcctgcatttgcaggcttcttatttttatggcgcgccgcattcacttcttttctatataaatatgagcgaagcgaataagcgtcggaaaagcagcaaaaagtttcctttttgctgttggagcatgggggttcagggggtgcagtatctgacgtcaatgccgagcgaaagcgagccgaagggtagcatttacgttagataaccccctgatatgctccgacgctttatatagaaaagaagattcaactaggtaaaatcttaatataggttgagatgataaggtttataaggaatttgtttgttctaatttttcactcattttgttctaatttcttttaacaaatgttcttttttttttagaacagttatgatatagttagaatagtttaaaataaggagtgagaaaaagatgaaagaaagatatggaacagtctataaaggctctcagaggctcatagacgaagaaagtggagaagtcatagaggtagacaagttataccgtaaacaaacgtctggtaacttcgtaaaggcatatatagtgcaattaataagtatgttagatatgattggcggaaaaaaacttaaaatcgttaactatatcctagataatgtccacttaagtaacaatacaatgatagctacaacaagagaaatagcaaaagctacaggaacaagtctacaaacagtaataacaacacttaaaatcttagaagaaggaaatattataaaaagaaaaactggagtattaatgttaaaccctgaactactaatgagaggcgacgaccaaaaacaaaaatacctcttactcgaatttgggaactttgagcaagaggcaaatgaaatagattgacctcccaataacaccacgtagttattgggaggtcaatctatgaaatgcgattaagggccggccagtgggcaagttgaaaaattcacaaaaatgtggtataatatctttgttcattagagcgataaacttgaatttgagagggaacttagatggtatttgaaaaaattgataaaaatagttggaacagaaaagagtattttgaccactactttgcaagtgtaccttgtacctacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaacttcaggtttgtctgtaactaaaaacaagtatttaagcaaaaacatcgtagaaatacggtgttttttgttaccctaagtttaaactcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcagggccccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgatcgggccccctgcagg

SEQ ID NO: 6 - DNA, 44 bp, Artificial Sequence
  Primer
ataaaagattcggatactaatgaactattaaagataaagcaagg

SEQ ID NO: 7 - DNA, 43 bp, Artificial Sequence
  Primer
ctgaattagttggttttcctttagtattgtgtaccgtatatcc

SEQ ID NO: 8 - DNA, 45 bp, Artificial Sequence
  Primer
gtctaatgaagatgatatcgatatattaggcatagctaaggatgg

SEQ ID NO: 9 - DNA, 44 bp, Artificial Sequence
  Primer
ccttttctatatatctttccatctacaacttctatagtttctcc

SEQ ID NO: 10 - DNA, 41 bp, Artificial Sequence
  Primer
catctgtatacatataactgacatctgtctatgtgctgtcc

SEQ ID NO: 11 - DNA, 42 bp, Artificial Sequence
  Primer
caccttcaagaaaattatattataatctgacatttttacctc

SEQ ID NO: 12 - DNA, 46 bp, Artificial Sequence
  Primer
gatagaaaatagataatatgctaaaagcaaaaactaatttagagcc

SEQ ID NO: 13 - DNA, 44 bp, Artificial Sequence
  Primer
ggaaaaatatattactttggtaataactcaaaagcagttactgg

SEQ ID NO: 14 - DNA, 29 bp, Artificial Sequence
  Primer
ggtattgctctactggcatttattttatg

SEQ ID NO: 15 - DNA, 31 bp, Artificial Sequence
  Primer
cttataatcatcaggtgttctagctaattgg

SEQ ID NO: 16 - DNA, 39 bp, Artificial Sequence
  Primer
ggatccgggcccggaaagatgtaaatagtgattataatg

SEQ ID NO: 17 - DNA, 35 bp, Artificial Sequence
  Primer
ggatccgggcccctcttaaatactttcctagttcc

SEQ ID NO: 18 - DNA, 40 bp, Artificial Sequence
  Primer
gacggatttcacatttgccgttttgtaaacgaattgcagg

SEQ ID NO: 19 - DNA, 40 bp, Artificial Sequence
  Primer
agatcctttgatcttttctacggggtctgacgctcagtgg

SEQ ID NO: 20 - DNA, 53 bp, Artificial Sequence
  Primer
ttttttgacgtcggtaaaataaaaggagattttaatgacagcaatttaatggg

SEQ ID NO: 21 - DNA, 47 bp, Artificial Sequence
  Primer
ccatgcaacctccattattacatctagtattaataagtccggttgtg

SEQ ID NO: 22 - DNA, 44 bp, Artificial Sequence
  Primer
gatgtaataatggaggttgcatggagtagaggaaaagttgacac

SEQ ID NO: 23 - DNA, 58 bp, Artificial Sequence
  Primer
ttttttgacgtcctccaacattatcaattattagtatattattttcagttaatatccc

SEQ ID NO: 24 - DNA, 1601 bp, Artificial Sequence
  Construct
ttttttgacgtcggtaaaataaaaggagattttaatgacagcaatttaatgggtaatttctcaaataattcagagctaggtataagtggtaatattacagaaaaccataataaagagtttaatgtagcaaataaagaaaagcaattaatagaagttggaaggccgcaagatgtaaaaataggagatgcagtaattctttttgaggataaaaacaaaaatataacaagctatgatataaaaatagaaagtatagtatatgataaaggaaattatagagatatggtaataaaagtagtagatgacaagttattggaatacacaggaggtatcgtacaggggatgagtggagctccaataatacaaaataataaaattattggtgcaataactcatgtttttagagataatccgaaaaaaggatatggtatttttatagatgaaatgataaaattgtaggtgaggcattaaaaaattttattattttatcaattatctaggaggaatataattttggagtgtcgaatatgctttagagtagataattaggaagcaattgtgtaaaaagtttagttttctgtaataagaagatgttttttaatggggggatttttagtggaaaaaatcaaaatagttttagcagatgacaataaggatttttgtcaggtattaaaagagtatttgtctaatgaagatgatatcgatatattaggcatagctaaggatggaattgaagcattagacttagtaaaaaagacacaaccggacttattaatactagatgtaataatggaggttgcatggagtagaggaaaagttgacacaataaatcaattatttggatatacggtacacaatactaaaggaaaaccaactaattcagaatttatagcaatgattgctgataaattaagactagaacatagtatggttaaataaacaagacataaaaagtaaggctttttaattaaggcattggctataaatgcgtattacaagcagcgaaacggtataaccactagggttataccgtttcgctattttaataaatataaaaattttctttattatttgcttactatatcaatataataattttattatactatggatatagtatgtgtctttacaagttgtaaactgacagtggtttattttttaatataaatattgactttgatgcaggtaaactttgtatttttaagcgtattgtggaatatgttaaataaaaaaatgatgaaatatagtattgtaaatgccaaagatgcaaaacaaacttaaaacatttattttattgttaagtaatgctataatataatgtgattttaataatgatagtggaggtttaaatatgagagtcgaggcccctataaaagtagatcgaaaaaccaaaaaacttgctaaaagagttgaaagtggggaaatagcagttataaatcatatagacatagatgaagttgctgcaaactctttagtagaagctaaaataaaacttgtcataaatgcggctccttctataagtggtaggtatcccaataaaggtccagggatattaactgaaaataatatactaataattgataatgttggaggacgtcaaaaaa

SEQ ID NO: 25 - DNA, 56 bp, Artificial Sequence
  Primer
ttttttgacgtctttccctacccctggaattttttgtagttctcccatacttcacc

SEQ ID NO: 26 - DNA, 49 bp, Artificial Sequence
  Primer
cagctatccccttagagcttccttttctttcattactaaattcgttacc

SEQ ID NO: 27 - DNA, 49 bp, Artificial Sequence
  Primer
ggaagctctaaggggatagctgtagagaaaattaattaatattgttttg

SEQ ID NO: 28 - DNA, 56 bp, Artificial Sequence
  Primer
ttttttgacgtcgtatattactttatgcctgatactgctatggctgcagctggtgg

SEQ ID NO: 29 - DNA, 1013 bp, Artificial Sequence
  Primer
ttttttgacgtctttccctacccctggaattttttgtagttctcccatacttcaccttctttctgatatattatttttgtattatacttagtaccagatattttttattatagttaatatttaatttttattatatcactttatttatgctctttcatctatctatattttaccacctctaaagtactgaatcatttaattacatcataatatagttttatacaaataaaatactttatgtttcatttaatatataaaattcaccttcaagaaaattatattataatctgacatttttacctcatttttcaaaatatattgaatcttcttgatttattttgtaaaattatgcttaggggaaatatattttaggaaaatatgaatatataatttttagtcaactagttattttaagtttttaaattttaaaataaaatatatctaataaaagggagattgtattatgttttctaaaaaaaatgagggtaacgaatttagtaatgaaagaaaaggaagctctaaggggatagctgtagagaaaattaattaatattgttttgtattatagttaatattttatattatagtcaatatgtttaaagatgtttttataattgcaaataaacagttacaaggctctaaattagtttttgcttttagcatattatctattttctatcaactattaattatttagtattaatatttccatatatgaattttattataaaatagtcaagaataatagattattaaatgatagaaaaattttaactaaaagtcatgtattacaataacacatgacttttaattaaatctcaatatttattatataaaaataatttctgagtatcacaggaataattttttgtcaaacatatattttagccatatatcccaggggcttttactccatcaacaccaaagaaatatataacaccatcaatctcgaaaagtccaccagctgcagccatagcagtatcaggcataaagtaatatacgacgtcaaaaaa

SEQ ID NO: 30 - DNA, 56 bp, Artificial Sequence; identical to SEQ ID NO: 25
  Primer

SEQ ID NO: 31 - DNA, 43 bp, Artificial Sequence
  Primer
cacacctaaaataaatgccagtagagcaatatcctttgtgctc

SEQ ID NO: 32 - DNA, 45 bp, Artificial Sequence
  Primer
ggcatttattttaggtgtgttttttggcaatatatcctcaccagc

SEQ ID NO: 33 - DNA, 52 bp, Artificial Sequence
  Primer
ttttttgacgtctttctctacagctatccctggtatggttatttttccaccc

SEQ ID NO: 34 - DNA, 1147 bp, Artificial Sequence
  Construct
ttttttgacgtctttccctacccctggaattttttgtagttctcccatacttcaccttctttctgatatattatttttgtattatacttagtaccagatattttttattatagttaatatttaatttttattatatcactttatttatgctctttcatctatctatattttaccacctctaaagtactgaatcatttaattacatcataatatagttttatacaaataaaatactttatgtttcatttaatatataaaattcaccttcaagaaaattatattataatctgacatttttacctcatttttcaaaatatattgaatcttcttgatttattttgtaaaattatgcttaggggaaatatattttaggaaaatatgaatatataatttttagtcaactagttattttaagtttttaaattttaaaataaaatatatctaataaaagggagattgtattatgttttctaaaaaaaatgagggtaacgaatttagtaatgaaagaaaaggaagctctaagaaaataattaaattctttaagagcacaaaggatattgctctactggcatttattttaggtgtgttttttggcaatatatcctcaccagcttgttctgaagaccatgaggaggtcatttctaatcaaacatcagttatagattctcaaaaaacagaaatagaaactttaaatagcaaattgtctgatgctgaaccatggttcaaaatgaaagacgacgaaaagaaagctattgaagctgaaaatcaacgtaaagctgaagaagctaaaaaggctgaagaacaacgtaaaaaagaagaagaagagaagaaaggatatgatactggtattacttatgaccaattagctagaacacctgatgattataagtacaaaaaggtaaaatttgaaggtaaggttattcaagttattgaagatggtgatgaggtgcaaataagattagctgtgtctggaaattatgataaggtcgtactatgtagttataaaaaatcaataactccttcaagagtgttagaggatgattacataactataagaggtataagtgctggaactataacttatgaatcaactatgggtggaaaaataaccataccagggatagctgtagagaaagacgtcaaaaaa

SEQ ID NO: 35 - DNA, 1025 bp, Artificial Sequence
  Construct
gtttaaacaagatgtaaatagtgattataatgttaatgttttttatgatagtaatgcatttttgataaacacattgaaaaaaactgtagtagaatcagcaataaatgatacacttgaatcatttagagaaaacttaaatgaccctagatttgactataataaattcttcagaaaacgtatggaaataatttatgataaacagaaaaatttcataaactactataaagctcaaagagaagaaaatcctgaacttataattgatgatattgtaaagacatatctttcaaatgagtattcaaaggagatagatgaacttaatacctatattgaagaatccttaaataaaattacacagaatagtggaaatgatgttagaaactttgaagaatttaaaaatggagagtcattcaacttatatgaacaagagttggtagaaaggtggaatttagctgctgcttctgacatattaagaatatctgcattaaaagaaattggtggtatgtatttagctgttgctatgttaccaggaatacaaccagacttatttgagtctatagagaaacctagttcagtaacagtggatttttgggaaatgacaaagttagaagctataatgaaatacaaagaatatataccagaatatacctcagaacattttgacatgttagacgaagaagttcaaagtagttttgaatctgttctagcttctaagtcagataaatcagaaatattctcatcacttggtgatatggaggcatcaccactagaagttaaaattgcatttaatagtaagggtattataaatcaagggctaatttctgtgaaagactcatattgtagcaatttaatagtaaaacaaatcgagaatagatataaaatattgaataatagtttaaatccagctattagcgaggataatgattttaatactacaacgaatacctttattgatagtataatggctgaagctaatgcagataatggtagatttatgatggaactaggaaagtatttaaggtttaaac

(19) United States
(12) Patent Application Publication
(10) Pub. No.: US 2012/0100616 A1
(43) Pub. Date: Apr. 26, 2012

(54) METHOD OF DOUBLE CROSSOVER HOMOLOGOUS RECOMBINATION IN CLOSTRIDIA

(75) Inventors: Stephen Thomas Cartman, Nottinghamshire (GB); Nigel Peter Minton, Nottinghamshire (GB)

(73) Assignee: THE UNIVERSITY OF NOTTINGHAM

(21) Appl. No.: 13/145,742

(22) PCT Filed: Jan. 21, 2010
(86) PCT No.: PCT/GB10/50084

§ 371 Date: Jan. 10, 2012

(30) Foreign Application Priority Data
Jan. 22, 2009 (GB) 0901001.8

(51) Int. Cl. C12N 15/74; C12N 1/21

(52) U.S. Cl. 435/471; 435/252.3; 435/320.1

(57) ABSTRACT

The invention relates to a method of double crossover homologous recombination in a host Clostridia cell comprising: a first homologous recombination event between a donor DNA molecule and DNA of the host cell to form a product of the first recombination event in the host cell, wherein the donor DNA molecule comprises a codA gene and at least two homology arms; and a second recombination event within the product of the first homologous recombination event, thereby to form a product of the second homologous recombination event in the host cell which is selectable by the loss of the codA gene; and a related vector and altered host cell.

The present invention relates to methods for modifying the nucleic acid of Clostridia cells, in particular, by using double crossover homologous recombination.

In order to undertake therapeutic target discovery in pathogens (ie, C. difficile), or to more effectively exploit the medical or industrial properties of beneficial strains (cancer or biofuel production), there is a requirement to be able to effectively and reproducibly manipulate microbial genomes at predetermined locations. For example, specific alterations may be made to a microbial genome in order to ablate (‘knock-out’) or alter an endogenous cellular function, or to expand function by adding one or more exogenous activities (‘knock-in’).

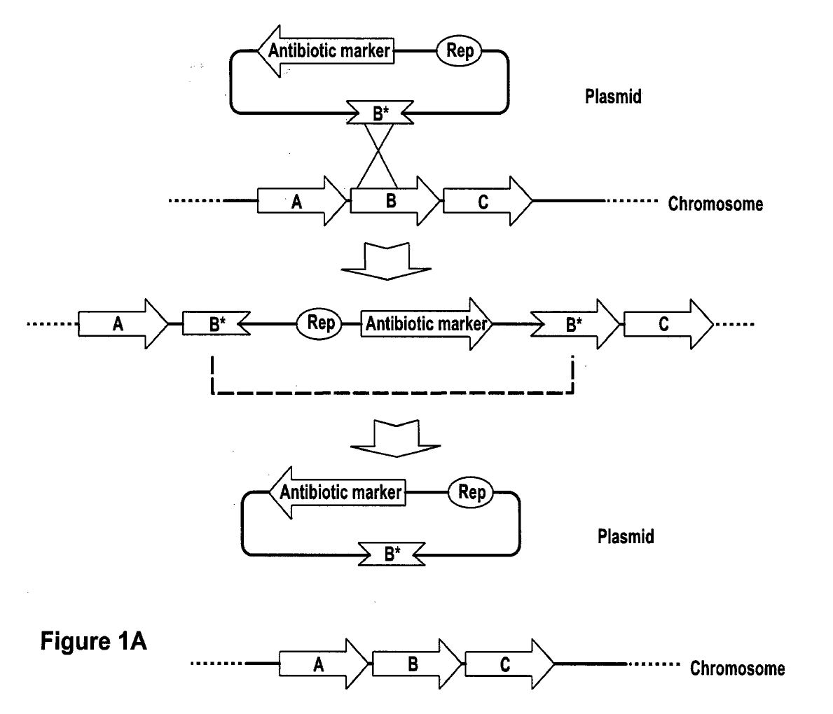

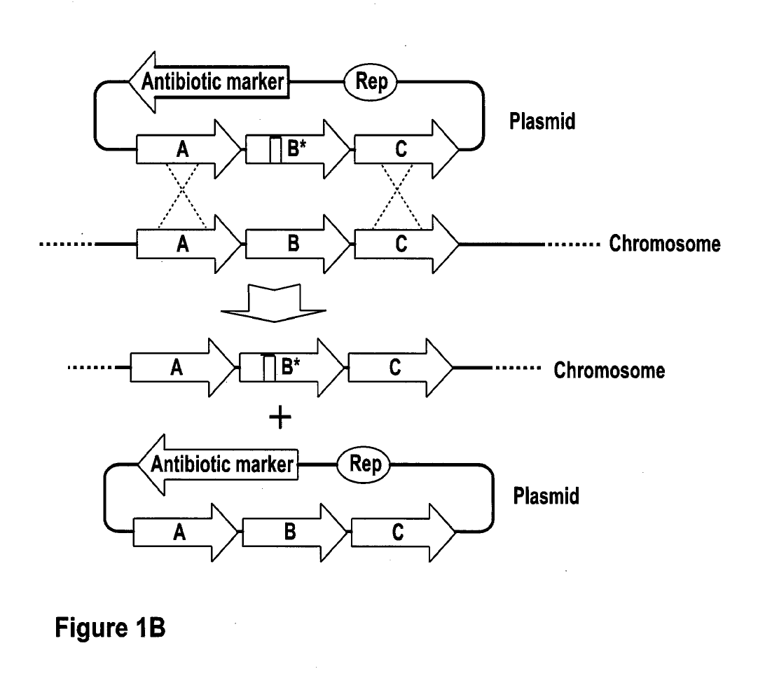

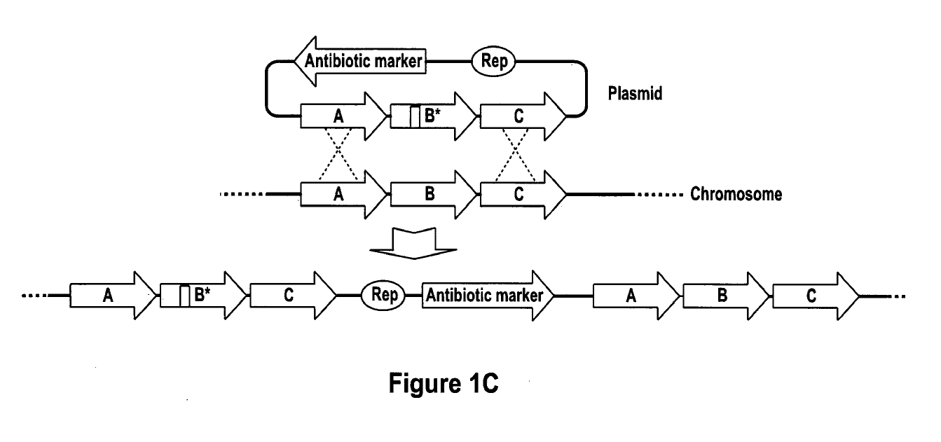

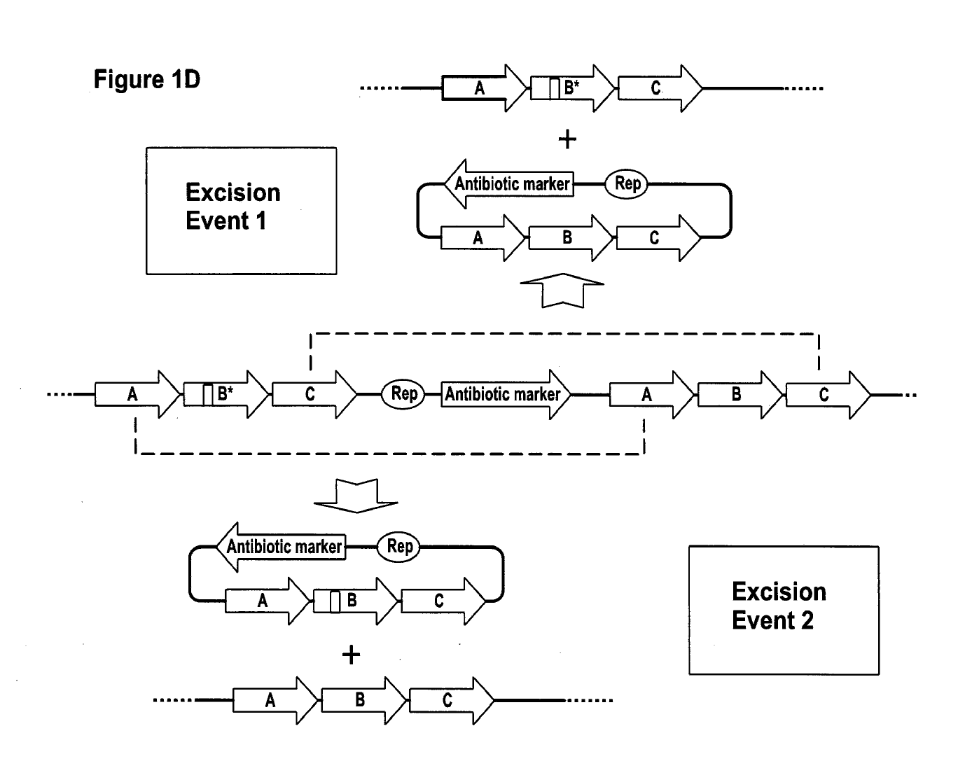

The host-cell recombination machinery is often key in genome manipulation procedures. In essence, two independent DNA molecules within a cell may ‘recombine’ via a Campbell-like mechanism to form a single DNA molecule, provided that they share a region of common DNA sequence (ie. a region of homology). Therefore, an extrachromosomal element (eg. a plasmid) introduced into a target host organism may ‘integrate’ into the host-cell genome to yield a ‘single cross-over integrant’. In order for a single cross-over event to inactivate a host-cell gene, the introduced DNA needs to encompass a central portion of the gene so that the extrachromosomal element integrates into the middle of the target gene, thus interrupting the coding sequence (FIG. 1A, step 1). It is noteworthy that single-cross over integrants are not genetically stable, as the regions of DNA sequence homology which facilitated the initial recombination event remain following integration. Therefore, a second recombination event can occur which results in excision of the extrachromosomal element and restoration of the target gene, essentially a reversing of the process (FIG. 1A, step 2).

It is possible to generate a genetically stable mutant via homologous recombination by modifying the design of the homologous DNA sequence in the extrachromosomal element (ie. the knock-out cassette). If the knock-out cassette is constructed such that the desired modification(s) is/are made to the target DNA sequence (i.e. deletion, insertion or alteration—indicated by * in FIG. 1B) and then flanked by DNA sequence which is homologous to that on either side of the target sequence (ie. homology arms), then a ‘double cross-over’ event can occur whereby two independent homologous recombination events happen, one in each homology arm (FIG. 1B). The process of a double cross-over event is often referred to as ‘allele exchange’ because the overall result is that the modified allele introduced on the extrachromosomal element is actually exchanged for the wild-type allele present in the host-cell genome.

Because of the low frequency with which recombination events occur (often estimated to be ≦1×10⁻⁶), the likelihood of detecting a double cross-over recombinant in which two independent cross-overs have occurred (ie., in FIG. 1B) is extremely low. Therefore, it is common practice to generate double cross-over mutants in a step-wise fashion, by isolating a single cross-over integrant in the first instance (FIG. 1C) and then subsequently isolating the double cross-over integrant (FIG. 1D).

As a result of integration, there exists homologous regions of DNA within the chromosome flanking the inserted plasmid, ie., in the example illustrated in FIG. 1 the genes A B & C. Homologous recombination between these duplicated regions will result in excision of the intervening DNA. If recombination occurs between a DNA region upstream of the mutation present in gene B, then the copy of gene B that will remain in the chromosome following plasmid excision is the original wild-type gene (Excision Event 2 in FIG. 1D). If however, recombination occurs between the region downstream of the mutation in gene B and the duplicated region, then the copy of gene B that will remain in the chromosome following plasmid excision is the mutated gene (Excision Event 1 in FIG. 1D).

Although the frequency with which a recombination event occurs is low, a single crossover integrant can be preferentially selected over a wild-type non-integrant cell, provided that the integrant has a growth advantage. In the most extreme case, if the plasmid replicon (Rep) is deficient (ie. completely non-functional), then when antibiotic resistance is encoded on the plasmid, the only way antibiotic resistant cells can arise is if the plasmid integrates into the host cell genome. Similarly, if the Rep region is defective (ie. functional but inferior to the host cell chromosomal replicon) then plasmid replication will be the limiting factor in terms of growth rate in the presence of antibiotic. Therefore, cells in which the plasmid integrates into the host cell genome will possess a growth advantage in the presence of antibiotic. Consequently, a single cross-over integrant can be selected under appropriate antibiotic selection, provided that the efficiency of the plasmid replicon used is suitably inferior to the host cell chromosomal replicon.

Having derived a single cross-over integrant, a practical problem arises in terms of being able to detect the rare second recombination event that leads to plasmid excision. In the scheme illustrated in FIG. 1D, such events are not selectable. They, therefore, must be detected by appropriate screening. The low frequency of occurrence makes this prohibitively time consuming.

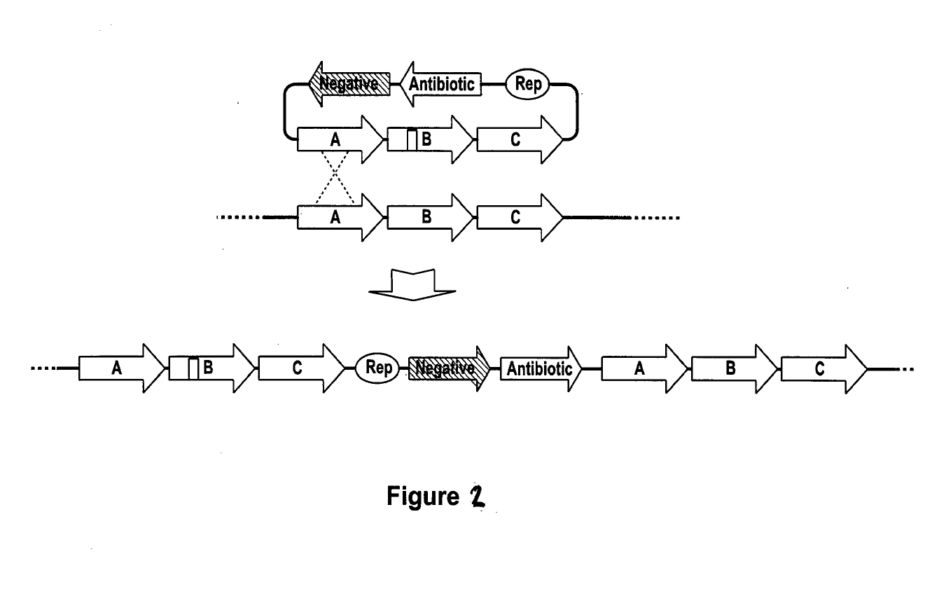

One way around this problem is to use a negative or counter selection marker. Such a marker may be included on the plasmid backbone along with the antibiotic resistance marker (FIG. 2). The marker is, therefore, incorporated into the chromosome as a consequence of integration of the plasmid by single cross-over recombination.

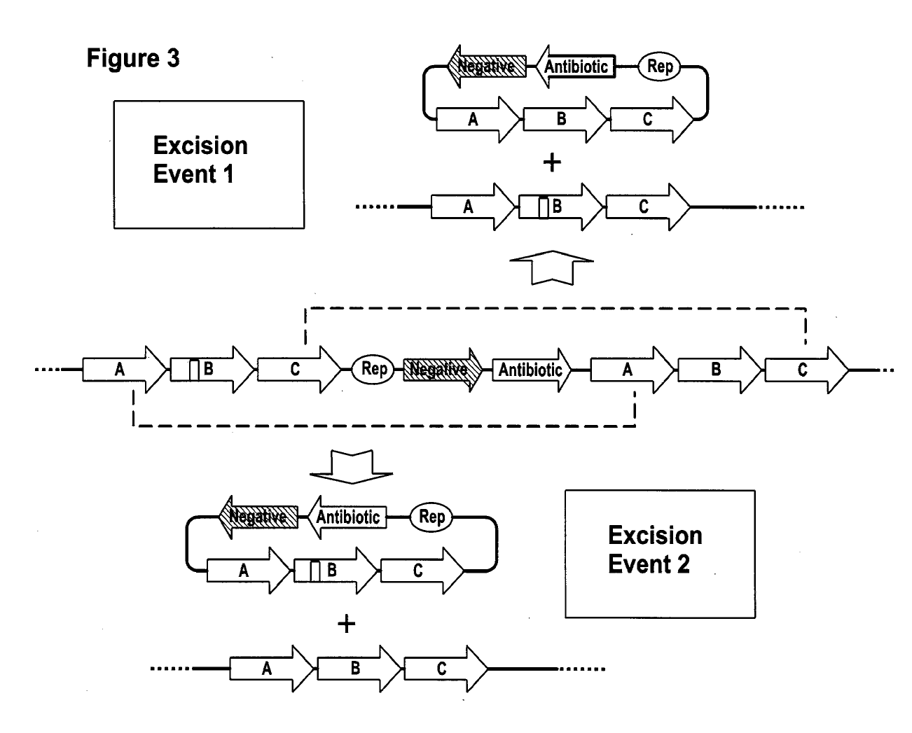

A feature of a negative selection marker is that under a specific defined condition its presence has a detrimental effect on the cell, most obviously causing cell death or preventing/inhibiting cell growth. Thus, by plating out the integrant generated in FIG. 2 under the non-permissive condition, the only cells that can survive/grow are those that have lost the negative selection marker due to plasmid excision (FIG. 3). Cells that survive may be subsequently screened for the presence of the desired excision event (FIG. 3).

The most commonly used negative selection marker is sacB of Bacillus subtilis. When introduced into heterologous hosts (principally Gram-negative bacteria) it causes lethality in the presence of exogenous sucrose (Kaniga et al., (1991) Gene 109:137-141).

In the case of Clostridia, no such equivalent heterologous gene for use as a negative selection marker has been described.

The class Clostridia includes the orders Clostridiales, Halanaerobiales and Thermoanaerobacteriales. The order Clostridiales includes the family Clostridiaceae, which includes the genus Clostridium. Clostridium is one of the largest bacterial genera. It is composed of obligately anaerobic, Gram-positive, spore formers. In recent years, the complete genome sequences of all of the major species of Clostridium have been determined from at least one representative strain, including C. acetobutylicum, C. difficile, C. botulinum and C. perfringens. C. acetobutylicum, together with other benign representatives, has demonstrable potential as a delivery vehicle for therapeutic agents directed against cancer. Certain members of the class may be employed on a commercial scale for the production of chemical fuels, eg, C. thermocellum and C. acetobutylicum. However, the genus has achieved greatest notoriety as a consequence of those members that cause disease in humans and domestic animals, eg, C. difficile, C. botulinum and C. perfringens. Despite the tremendous commercial and medical importance of the genus, progress either towards their effective exploitation, or on the development of rational approaches to counter the diseases they cause, has been severely hindered by the lack of a basic understanding of the organisms' biology at the molecular level. This is largely a consequence of an absence of effective genetic tools.

One approach adopted has been to adapt the B. subtilis method described by Fabret et al. (Mol Microbiol (2002) 46:25-36) for use in C. acetobutylicum. This method relies on the sensitivity of host cells to 5-fluorouracil, as a consequence of its conversion to 5-fluoro-dUMP by uracil-phosphoribosyl-transferase, encoded by the upp gene. Fabret et al. (2002) deleted the upp gene from the genome of C. acetobutylicum, enabling them to use upp on the knock-out vector as a negative selection marker. Thus, loss of the upp gene following excision of the plasmid can be selected by the isolation of 5-fluorouracil resistant colonies. Whilst this method is extremely powerful, it requires the use of a mutant host strain, that is, a strain mutant for the app gene. The advisability of using a strain that is mutated in upp for virulence studies in a pathogen is questionable.

Generally, when studying the biology of an organism at the molecular level, it is preferable to start with a wild-type background, as any recombinant strains generated can be compared directly back to the wild-type parental strain (which in the case of a pathogen such as C. difficile may also be a clinically isolated strain). As the wild-type strain and the recombinant strain generated are isogenic (except for the genetic modification deliberately introduced into the recombinant strain), any phenotypic differences between them can be directly attributed to the genetic modification made. The drawbacks of starting with a mutant strain include that there is labour involved in generating the initial ‘starting strain’. Another drawback is that the skilled man has to compare a single-mutant (parental) strain with a double mutant (descendent) strain in any experiment carried out, and then has to extrapolate back to glean the meaning for the original wild-type (clinical) strain. In this instance it is more difficult to say with certainty whether any phenotypic differences observed between the parental and the descendent strains are purely due to the secondary mutation carried by the descendent strain or whether there is a combinatory/synergistic effect between the primary and secondary mutations.

In this invention, the codA gene from E. coli is used as a heterologous negative, or positive, selection marker in Clostridia. Surprisingly, codA functions in Clostridia and makes a powerful negative, or positive, selection marker, thereby avoiding the need to always undertake manipulations in a mutant Clostridia strain, eg., a strain with a mutant gene.

The codA gene encodes cytosine deaminase which catalyses the conversion of cytosine to uracil, and ammonia. It can also catalyse the conversion of the innocuous ‘pro-drug’ 5-fluorocytosine (FC), into the highly cytotoxic drug, 5-fluorouracil (FU). FU can exert toxicity in two ways, i) by inhibiting the essential pyrimidine biosynthesis pathway and/or ii) by incorporation into DNA and RNA molecules.

The codA gene has been used as a negative selection marker for genetic manipulation of mammalian and plant cells. It has never been used, in the classic sense, as a negative selection marker in prokaryotes (ie. for making specifically targeted, defined mutations).

According to a first aspect, the invention provides a method of double crossover homologous recombination in a host Clostridia cell comprising:

a first homologous recombination event between a donor DNA molecule and DNA of the host cell to form a product of the first recombination event in the host cell, wherein the donor DNA molecule comprises a codA gene and at least two homology arms; and
a second recombination event within the product of the first homologous recombination event, thereby to form a product of the second homologous recombination event in the host cell which is selectable by the loss of the codA gene.

Preferably the DNA of the host cell comprises a chromosome or a plasmid of the host cell, most preferably the DNA of the host cell comprises a chromosome of the host cell.

Preferably the donor DNA molecule further comprises a selectable allele.

Preferably, the codA gene functions in the method of the invention as a negative selection marker. Preferably, codA is the only negative selection marker used in the method of the invention.

Alternatively, the codA gene may function as a positive selection marker, in that it may be used to select for those cells which carry the gene (Wei and Huber (1996) The Journal of Biological Chemistry 271(7):3812-3816), rather than as a negative selection marker wherein cells which carry the gene are selected against.

The product of the first homologous recombination event is a single crossover integrant of the donor DNA molecule into the host DNA. The product of the first crossover event may not be uniform, but may comprise different molecular species depending on the location at which the donor DNA molecule integrates into the acceptor DNA molecule. Nevertheless, it may not be necessary to select between different first recombination products. It may be that the different molecular species in the product of the first recombination event can each give rise to the desired product of the second recombination event. Even in situations in which not all possible molecular species in the product of the first recombination event can give rise to the desired product of the second recombination event, it may be that undesired products occur so rarely that it is not necessary to select against them.

Preferably a selectable marker on the first donor DNA molecule is expressed upon integration into the host DNA. By screening for expression of the selectable marker, products of the first homologous recombination event may be isolated. Preferably, the donor DNA molecule is not efficiently replicated in the host cell, such that even if the selectable marker is expressed on the donor DNA molecule the levels of expression are insufficient to allow these cells to be identified in a screen for products of the first homologous recombination event based on expression of the selectable marker, or expression of the selectable marker on the unintegrated donor DNA results in colonies which grow markedly slower than those which are the product of the first homologous recombination event, thus the required cells can be easily distinguished. Products of the first homologous recombination event may alternatively, or additionally, be screened for by screening for expression of the codA gene upon integration of the donor DNA molecule into the host DNA. Such cells would be unable to grow on medium containing 5-fluorocytosine, which would be converted by the product of the codA gene into the highly cytotoxic drug, 5-fluorouracil.

The selectable marker encoded by the donor DNA molecule, which allows selection of integrants, may be an enzyme which detoxifies a toxin, such as an antibiotic resistance enzyme or a pro-drug converting enzyme; a fluorescent or coloured maker gene; a marker of auxotrophy; or any other suitable marker. Suitable selectable marker genes may encode resistance to antibiotics (eg., to tetracycline, erythromycin, neomycin, lincomycin, spectinomycin, ampicillin, penicillin, chloramphenciol, thiamphenicol, streptinomycin, kanamycin, etc), chemicals (eg., herbicides), heavy metals (eg., cadmium, mercury, selenium, etc.) and other agents (eg., UV, radiation), as well as genes that complement chromosomal defects in the recipient organism (eg., leuD, murA, manA). Typically the selectable marker gene confers a growth or survival advantage on a host cell in which the first recombination event has occurred. Preferably, the selectable marker gene is not retained in the product of the second recombination event. Suitably, this may be achieved by locating the selectable marker gene in the donor DNA molecule upstream of the homology arm providing the first site of recombination, or downstream of the homology arm providing the second site of recombination.

The method of the invention preferably includes the step of selecting for products of the first homologous recombination event. This may be achieved by (i) growing the cells under conditions in which cells with a selectable marker integrated into the host DNA have a growth advantage, and/or (ii) selecting for 5-fluorocytosine sensitivity conferred by the expression of the codA gene.

The method of the invention preferably includes the step of selecting for products of the second homologous recombination event, this may be achieved by growing the cells in medium containing 5-fluorocytosine, which is toxic to cells expressing the codA gene at a significant level. Any cells which have not undergone a second homologous recombination, and thus still have the codA gene integrated into the host DNA, will not be able to grow in the presence of 5-fluorocytosine, and only those cells which have excised the donor DNA will be able to grow.

Having identified products of the second homologous recombination event, PCR or other analytical tests may then be used to identify which of the surviving cells have excised the plasmid/donor DNA molecule in the desired manner, namely to introduce an alternative allele into the host DNA.

Preferably the donor DNA molecule further comprises an alternative allele which is introduced into the host DNA in the first homologous recombination event. The alternative allele is preferably retained in the host DNA following the second homologous recombination event. The alternative allele may introduce a mutation into the corresponding allele in the host DNA; this mutation may be an insertion, deletion or any other appropriate mutation. For example, the method of the invention may be used to inactivate a gene endogenous to the host DNA by introducing a functionless alternative allele into, or in place of, the endogenous gene.

Alternatively, or additionally, the alternative allele may be so called “cargo” DNA which is to be added to the host DNA. Cargo DNA may be selected to confer a desirable phenotype on the host cell, such as the ability to express a particular protein. There is no particular limitation on the selection of the cargo DNA. There is no particular limit to the size of the cargo DNA although, in practice, this will be limited by the size of the donor DNA molecule. Depending on the host cell, there may be a practical limit to the size of the donor DNA molecule that can be introduced. For example, in certain Clostridia, transformation of plasmids is poorly efficient and efficiency is reduced when the size of the plasmid is increased. The skilled person can readily determine experimentally an upper limit for the size of the cargo DNA, which may vary depending on the host cell and the donor DNA molecule. Suitably, cargo DNA of at least 1 bp may be introduced, preferably at least 1, 2, 3, 4, 5, 10, 15, 20, 50, 100, 1000, 10,000, 100,000 or 1,000,000 kb.

Cargo DNA may comprise genes or other genetic material from the same genus as the host cell, or from a different genus. Cargo DNA may also be entirely synthetic, or any combination of synthetic and natural genetic material. Genes may function in, for example, a catabolic pathway or a biosynthetic pathway.

Preferably, the donor DNA molecule comprises at least two homology arms, one homology arm providing for homologous recombination with the host DNA at a first site upstream of an alternative allele to be exchanged, and one homology arm providing for homologous recombination with the host DNA at a second site downstream of the alternative to be exchanged. The host DNA preferably comprises homology arms corresponding to the homology arms of the donor DNA molecule, and the allele to be exchanged with the alternative allele in the donor DNA molecule is located upstream of the first corresponding homology arm or downstream of the second corresponding homology arm. Homology arms provide for homologous recombination between the donor DNA molecule and the host DNA in the first recombination event, and within the product of the first recombination event in the second recombination event. The extent of homology between corresponding homology arms must be sufficient to allow homologous recombination to occur. Factors affecting whether homologous recombination can occur are the sequence identity between the corresponding homology arms and the base-pair size of the homology arms. Typically, at least 85% sequence identity is required between corresponding homology arms for homologous recombination to occur. Preferably, the sequence identity is at least 90%, more preferably at least 95%, still more preferably at least 98% and most preferably 100%. Typically, the size of each homology arm is at least 10 bp, more typically at least 20 bp, at least 40 bp, at least 75 bp, at least 100 bp, at least 200 bp, or at least 300 bp. There is no particular upper limit for the size of the homology arm although in practice this may be governed by the size of the donor DNA molecule, which must have at least two homology arms. A homology arm could be as large as 1 kb, or up to 2 kb, up to 5 kb, up to 10 kb, even up to 50 kb, 100 kb, 1 Mb, 5 Mb or 10 Mb.

As noted above, the product of the first recombination event may not be uniform, but may comprise different molecular species depending on the location at which the donor DNA molecule integrates into the host DNA. Each homology arm in the donor DNA molecule has a corresponding homology arm in the host DNA. The homology arm in the donor DNA molecule and the corresponding homology arm in the host DNA can be considered to be a pair.

The first recombination event may occur by homologous recombination in either the first pair of homology arms, or the second pair of homology arms. Thus, typically, in some host cells the homologous recombination occurs at the first pair of homology arms and in others homologous recombination occurs at the second pair of homology arms, such that different molecular species of DNA are formed by the first recombination event. Both pairs of homology arms are present in the product of the first recombination event.

If a second recombination event occurs between the same pair of homology arms in which the first recombination event occurred, the donor DNA molecule will be recombined out, and the host DNA will be restored to its original form. In contrast, the desired product of the second recombination event is formed by homologous recombination between the pair of homology arms that did not recombine in the first recombination event. Thus, although both homology arms of the donor DNA molecule can provide for homologous recombination with the host DNA, it is to be understood that, for any particular donor DNA molecule, only one homology arm will homologously recombine with the host DNA, and the other homology arm will homologously recombine intramolecularly in the product of the first recombination event.

In particular embodiments, there may be more than two pairs of homology arms, for example there may be three pairs of homology arms. As in the case where there are two pairs of homology arms, only one homology arm will homologously recombine with the host DNA, and another homology arm will homologously recombine intramolecularly in the product of the first recombination event.

It will be understood that when a pair of homology arms undergo homologous recombination, the exact site of homologous recombination is unpredictable. If the pair are identical in DNA sequence, the products of homologous recombination are also identical in sequence, even though the exact site at which the integration occurs is unknown.

As noted above, even if the first recombination event can produce different products depending on where the donor DNA molecule integrates into the host DNA, it may not be necessary to select against host cells in which a particular first recombination event has occurred. It may be possible to favour the first recombination event occurring at a desired pair of homology arms, this can be achieved by making the desired homology arm in the donor DNA molecule longer than the other homology arm or arms in the donor DNA molecule. For example, the length of the homology arm at which the first recombination event is desired to occur may be up to about 1200 bp. Other homology arms in the donor DNA molecule may be about 300 bp to about 500 bp. The first recombination event occur may then occur more prevalently at the about 1200 bp pair of homology arms.

The donor DNA molecule may be any DNA molecule suitable for use in double crossover homologous recombination. Preferably, in the method of the invention, the donor DNA molecule is a plasmid, particularly a non-replicative plasmid, a replication-defective plasmid or a conditional plasmid. Alternatively, the donor DNA molecule may be linear or it may be a filamentous phage like M13. The skilled person can readily select a donor DNA molecule, such as a plasmid, which is suitable for use with a given host cell.

A non-replicative plasmid would include those plasmids which do not carry ‘machinery’ able to support the autonomous replication of the plasmid in the intended recipient host. Such plasmids, referred to as suicide vectors, designed for use in a Gram-positive host would include, for instance, plasmids based on the ColE1 replicon, but which lack replication functions derived from Gram-positive plasmids (eg., pMTL30, Wilkinson and Young (1994). Microbiology 140, 89-95).

A replication-defective plasmid would carry replication functions that function only inefficiently in the intended recipient host. Such plasmids would be characterised by their segregational instability in the intended host in the absence of any form of selective pressure. For instance, where such a plasmid carries a gene encoding antibiotic resistance, and cells are grown in media lacking that antibiotic, daughter cells would arise which have not received a replicative copy of that plasmid. Moreover, in the presence of the antibiotic, the growth rate of the cell population as a whole will be reduced, due to ineffective segregation of the antibiotic resistance gene. Many Gram-positive/E. coli shuttle vectors replicate poorly in their intended host. For instance, the majority of clostridial plasmids are segregationally unstable (Minton et al (1993) In “The Clostridia and Biotechnology”, ed. DR Woods, pp. 119-150, Butterworths-Heinemann), including plasmids based on the pIM13 replicon (Harris et al (2002) J. Bacteriol. 184, 3586-3597) and pIP404 and pCB102 (Purdy et al (2002) Molecular Microbiology 46, 439-52). Plasmids that replicate via a single-stranded deoxyribonucleic acid (ssDNA) intermediate by a rolling-circle mechanism are the most common family of Gram-positive plasmid. Vectors based on such plasmids are frequently segregationally unstable (Gruss and Ehrlich (1989) Microbiol Mol Biol Rev 53, 231-241). Other plasmids may be deliberately engineered to possess the required instability, such as the frame shift introduced into the repH gene of the pCB102 replicon (Davis (1998) “Regulation of botulinum toxin complex formation in Clostridium botulinum”, PhD Thesis Open University).

Conditional vectors represent those plasmids that cannot replicate under defined, non-permissive conditions. Examples of such vectors for E. coli include ColE1-derived plasmids, which do not replicate in polA mutants (Gutterson and Koshland (1983) Proc Natl Acad Sci USA. 80, 4894-4898; Saarilahti and Palva (1985) FEMS Microbiol Lett. 26, 27-33), a temperature-sensitive pSC101 replicon (Hamilton et al (1989) J Bacteriol 171, 4617-4622), and a phagemid-based vector (Slater et al (1993) J Bacteriol 175, 260-4262), Thermosensitive, pir-dependent, and repA-dependent broad-host-range plasmids for use in Gram-positive bacteria have been described (Biswas et al (1993) J. Bacteriol. 175, 3628-3635, Leenhouts et al (1996) Mol Gen Genet. 253, 217-224; Miller and Mekalanos (1988) J Bacteriol 170, 2575-2583).

Typically, a ‘suicide’/non-replicative plasmid requires high frequencies of DNA transfer in order for the rare recombination events to be detected; an ‘unstable’/replication-defective plasmid does not require high frequencies of DNA transfer, but instead relies upon the growth rate differential between plasmid replication and chromosome replication; a conditional plasmid does not require high frequencies of DNA transfer, and its replication rate can be decreased by a user-controlled variable such as temperature. Alternatively, the effective rate of replication of many plasmids in microorganisms can be decreased by culturing cells under conditions which promote plasmid loss, e.g, in phosphate- or sulphate-limited media in the case of E. coli (Jones et al (1980) Gen Genet. 180, 579-584; Caulcott et al (1987) J Gen Microbiol 133, 1881-1889) or magnesium-limited media in the case of Saccharomyces cerevisiae (O'Kennedy and Patching (1997) J Ind Microbiol Biotechnol 8, 319-325).

Where the host cell is a bacterium that is difficult to transform, it is convenient that the donor DNA molecule is a shuttle vector which allows for replication and propagation in a bacterial cell such as Escherichia coli and in the host cell. Additionally or alternatively, the donor DNA molecule may contain a region which permits conjugative transfer from one bacterial cell such as E. coli to a bacterial host cell. Methods of transformation and conjugation in Clostridia are provided in Davis, I, Carter, G, Young, M and Minton, NP (2005) “Gene Cloning in Clostridia”, In: Handbook on Clostridia (Durre P, ed) pages 37-52, CRC Press, Boca Raton, USA.

The host Clostridia cell may be a species of the genus Clostridium, which includes C. acetobutylicum, C. cellulolyticum, C. phytofermentans, C. thermocellum, C. beijerinckii, C. saccharobutylicum, C. saccharoperbutylacetonicum, C. difficile, C. botulinum, C. sporogenes, C. butyricum, and C. perfringens. Another preferred species of the class Clostridia is Thermoanaerobacterium saccharolyticum.

Preferably, the method of the invention further comprises the step of transforming a host Clostridia cell with a donor DNA molecule prior to the first homologous recombination event.

Preferably, the method of the invention comprises the further step of isolating the host cell comprising the product of the second homologous recombination event by virtue of the altered phenotype conferred by the loss of the codA gene, so as to provide an altered isolated host cell. The host cell may be altered by the introduction of the alternative allele. Thus, the invention provides a method of producing an altered host cell, the method comprising providing a host cell and carrying out the aforesaid method.

The invention therefore includes an altered host cell obtained by the method of the invention.

According to another aspect, the invention provides a vector (donor DNA molecule), such as a plasmid, comprising the codA gene, and at least two homology arms for the transformation of Clostridia cells. Preferably, the vector also comprises a selectable marker. The vector may also comprise a cloning site for inserting an alternative allele. The vector may also comprise an alternative allele.

Preferably the vector is non-replicative or a replication-defective in Clostridia cells.

Preferably the codA gene and the selectable marker, if present, are expressed when the vector in integrated into the DNA, preferably a chromosome, of a Clostridia cell.

Preferably the codA gene functions as a negative selection marker. Alternatively, the codA gene may funcation as a positive selection marker.

Preferably the codA gene allows cells which have recombined out the vector to be identified. Preferably the vector does not contain any further genes, in addition to the codA gene, in order to allow the selection of cells which have recombined out the vector.

According to any aspect of the invention, where appropriate, the donor DNA molecule may comprise a polynucleotide sequence selected from any of the group comprising SEQ ID NO: 2, SEQ ID NO: 3, SEQ ID NO: 4 and SEQ ID NO: 5.

According to another aspect of the invention, there is provided a vector comprising a sequence selected from any of SEQ ID NO: 2, SEQ ID NO: 3, SEQ ID NO: 4 and SEQ ID NO: 5.

The invention will now be described with reference to the following non-limiting Example and Figures.

FIG. 1A—illustrates schematically a prior art single crossover event;

FIG. 1B—illustrates schematically a prior art double crossover homologous recombination event (also referred to as allele exchange);

FIG. 1C—illustrates schematically the first recombination event of a double crossover homologous recombination event (prior art);

FIG. 1D—illustrates schematically the possible second recombination events of the double crossover homologous recombination event of FIG. 1C (prior art);

FIG. 2—illustrates schematically a first recombination event using a donor plasmid carrying a negative selection marker;

FIG. 3—illustrates schematically the possible second recombination events following the first recombination event of FIG. 2;

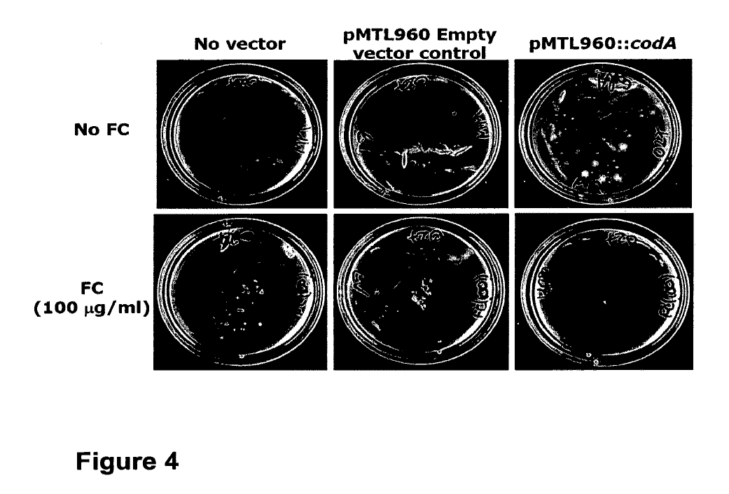

FIG. 4—shows the results of functionality testing of a codA cassette for counter selection of C. difficile on minimal growth medium with and without FC;

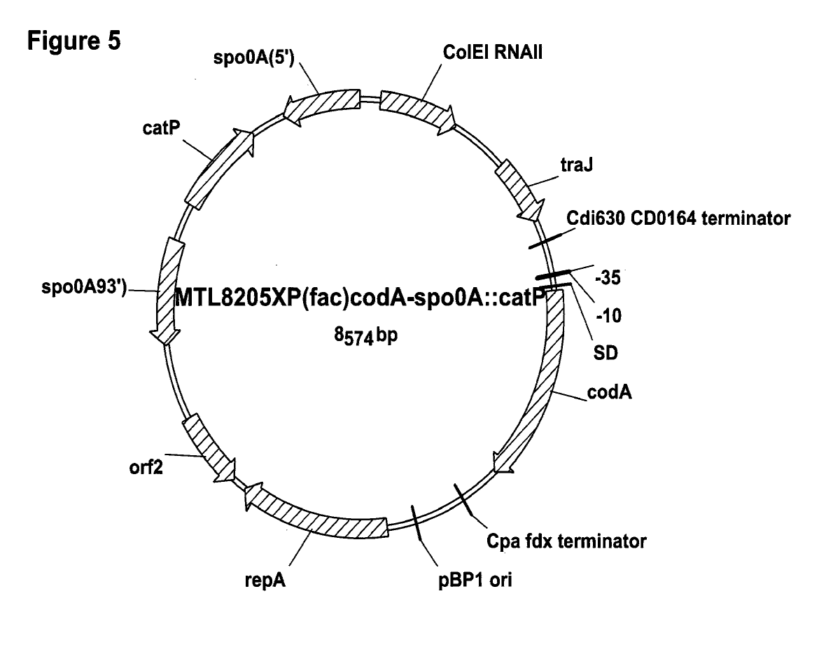

FIG. 5—shows a plasmid constructed for inactivating the spo0A gene of C. difficile by allele exchange. The codA negative selection cassette is flanked by terminators so that it is less susceptible to transcriptional read-through from other open reading frames in the plasmid.

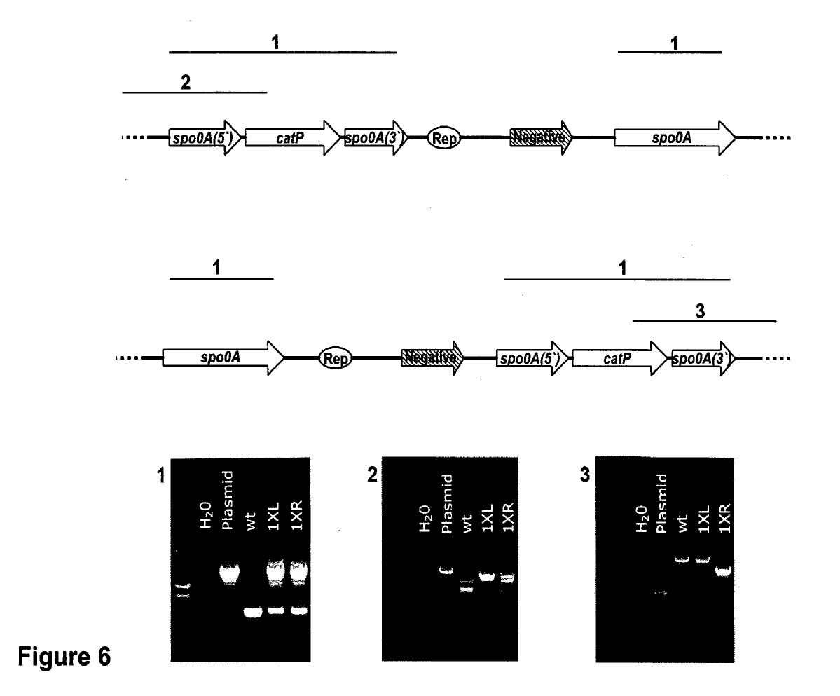

FIG. 6—shows the results of the screening of colonies for single cross-over integrants/products of the first recombination event. The top two illustrations depict the two possible outcomes of a single cross-over event when homologous recombination occurs in the left homology arm [spo0A(5′)] or the right homology arm [spo0A(3′)] of the spo0A knock-out cassette, respectively. The numbered lines indicate the regions of sequence amplified by PCR when forward (F) and Reverse (R) primer pairs are targeted as follows, 1. F-spo0A left homology arm/R-spo0A right homology arm; 2. F-upstream of spo0A left homology arm/R-catP sequence; 3. F-catP sequence/R-downstream of spo0A right homology arm. The lower illustration includes photographs of three gels (1, 2 and 3) showing the PCR results obtained when screening was carried out to show the products of PCRs designed to amplify regions 1, 2 and 3, respectively (ie. those depicted in the upper two illustrations). wt, wild-type C. difficile 027; 1XL, single cross-over integrant in which the homologous recombination event took place in the left homology arm of spo0A (ie. upper illustration); 1XR, single cross-over integrant in which the homologous recombination event took place in the right homology arm of spoOA (ie. middle illustration).

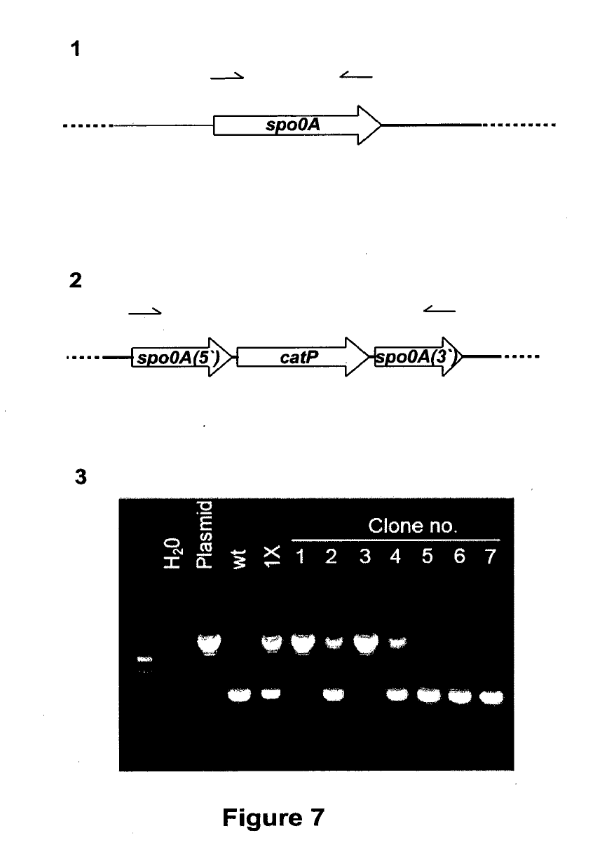

FIG. 7—shows the results of PCR screening of products of the double cross-over/products of the second recombination. Screening was done using primers which anneal in the homology arms of the spo0A knock-out cassette. The two expected outcomes, following the use of FC to select for double cross-overs/products of the second recombination event, which have lost the excised plasmid (and hence, have lost codA) are depicted in 1 and 2. PCR screening was carried out using primers which anneal to the left homology arm and the right homology arm of spo0A—indicated by the half arrows. The gel shows the results of PCR screening carried out for seven individual clones. Clones 1 and 3 gave rise to a larger PCR product only, indicating that they are double cross-over mutants in which catP has been inserted into spo0A. Clones 5, 6 and 7 gave rise to a smaller PCR product only, indicating that they are double cross-over mutants in which the second recombination event occurred in the same homology arm as the first. They are therefore wild-type revertants. Finally, clones 2 and 4 gave rise to both the smaller and the larger PCR product, the same as the single cross-over integrant control (1×). This suggests that these clones are single cross-overs with a spontaneous mutation which alleviates the effect of codA in the presence of FC.

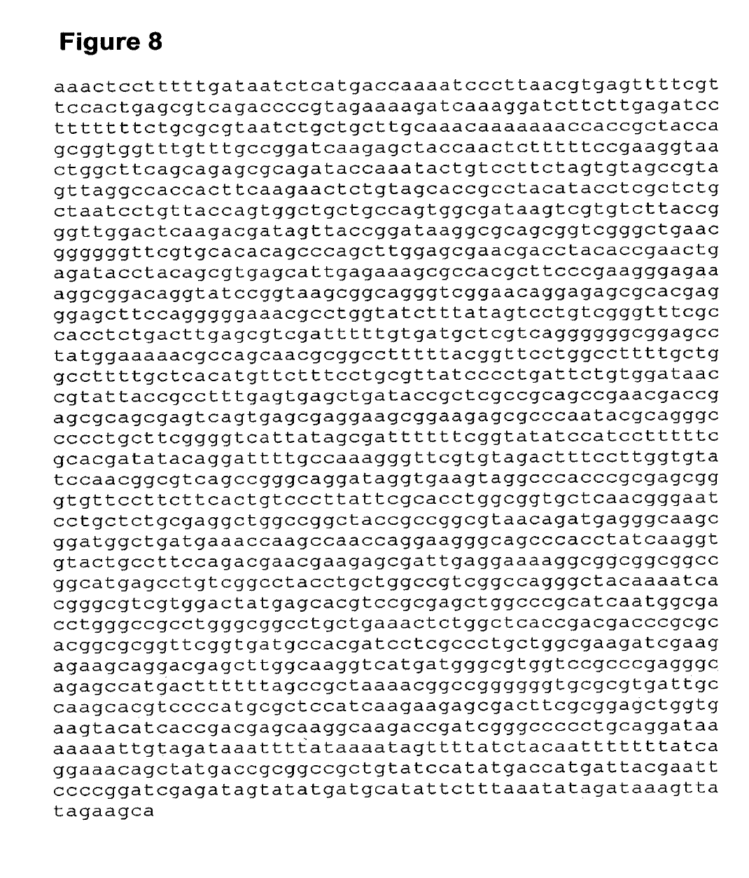

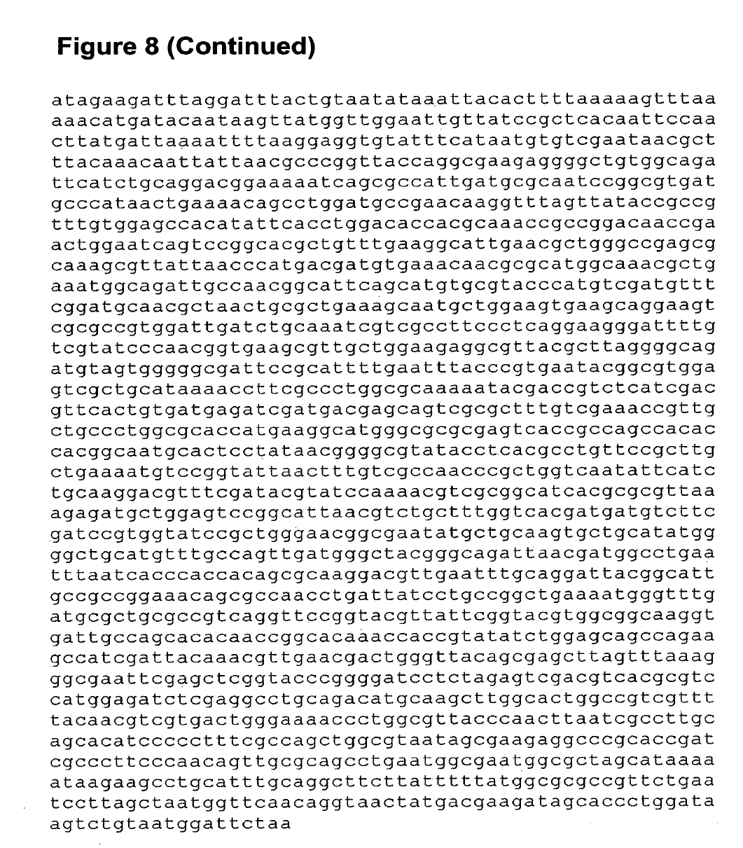

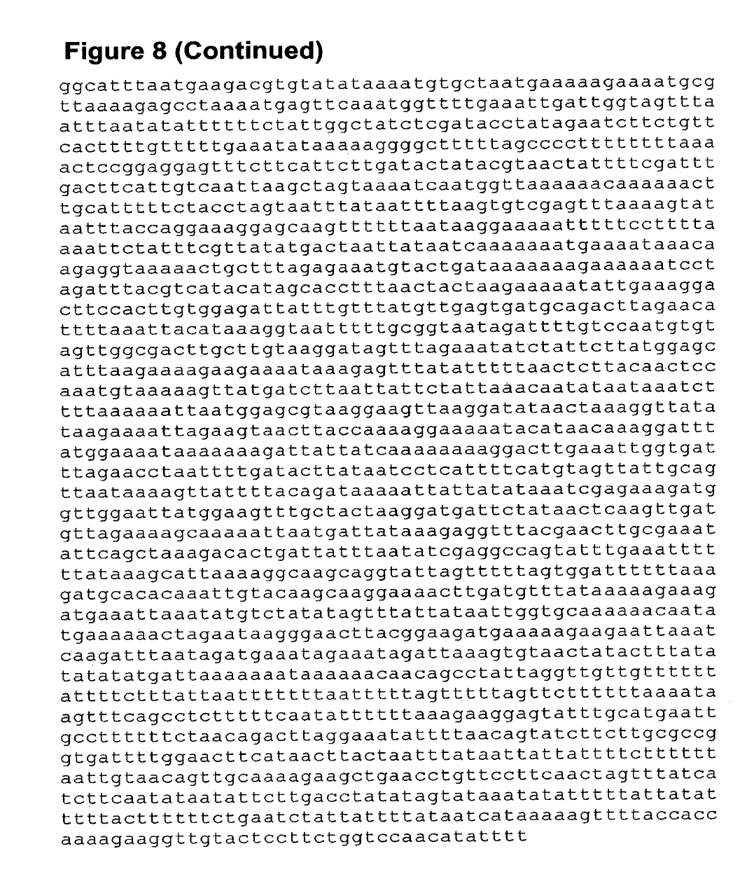

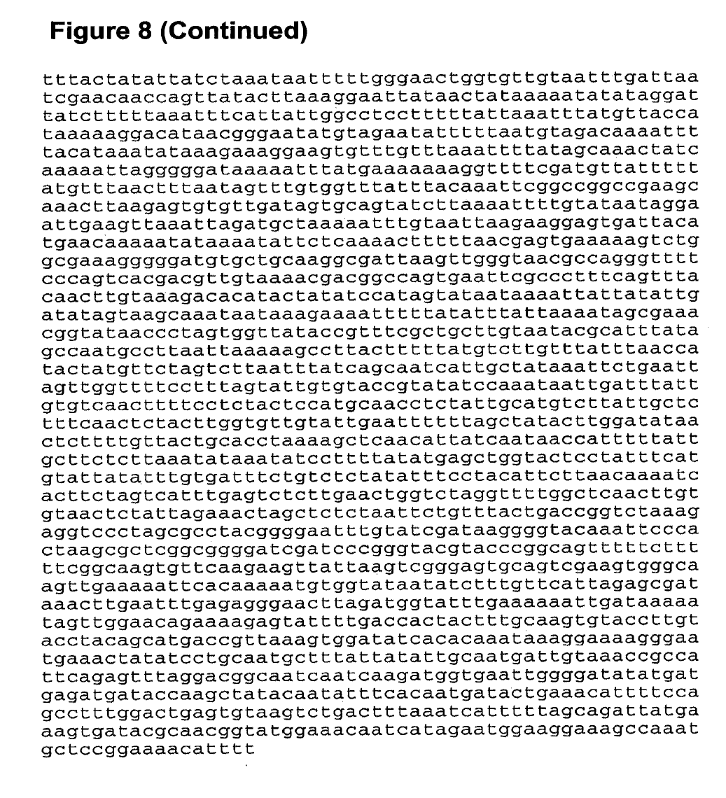

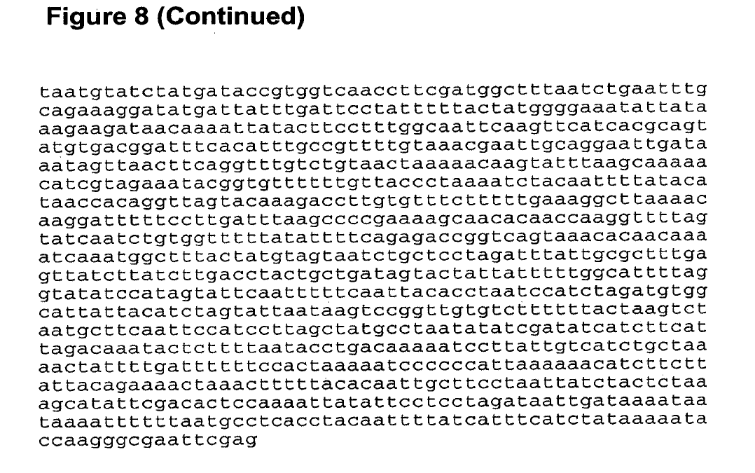

FIG. 8—is the DNA sequence of the plasmid of FIG. 5 (SEQ ID NO: 1).

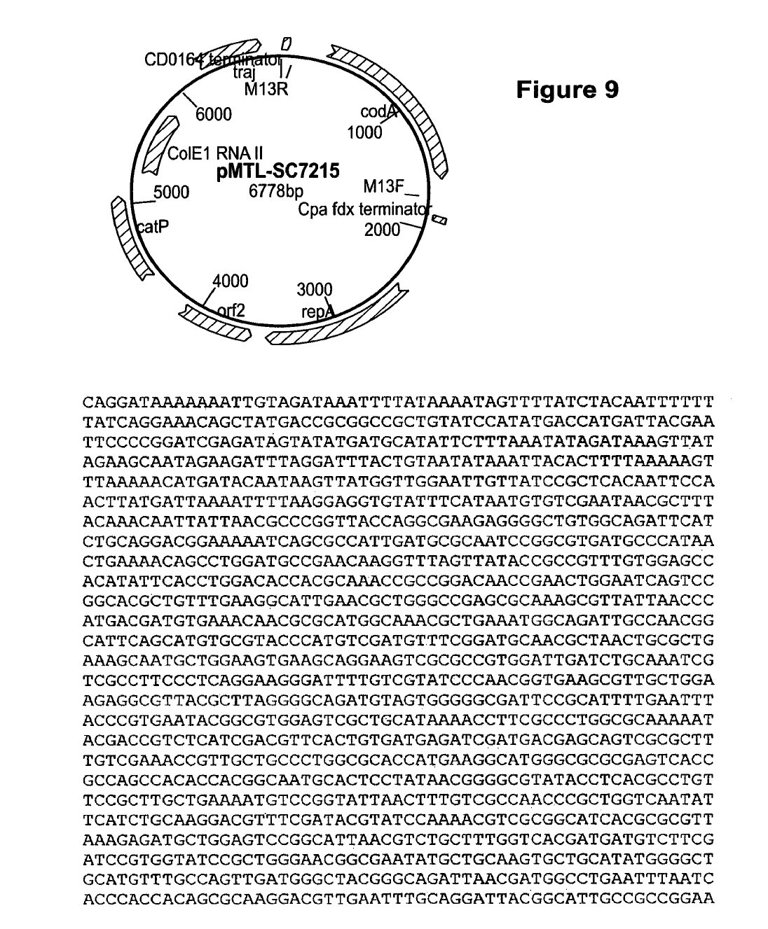

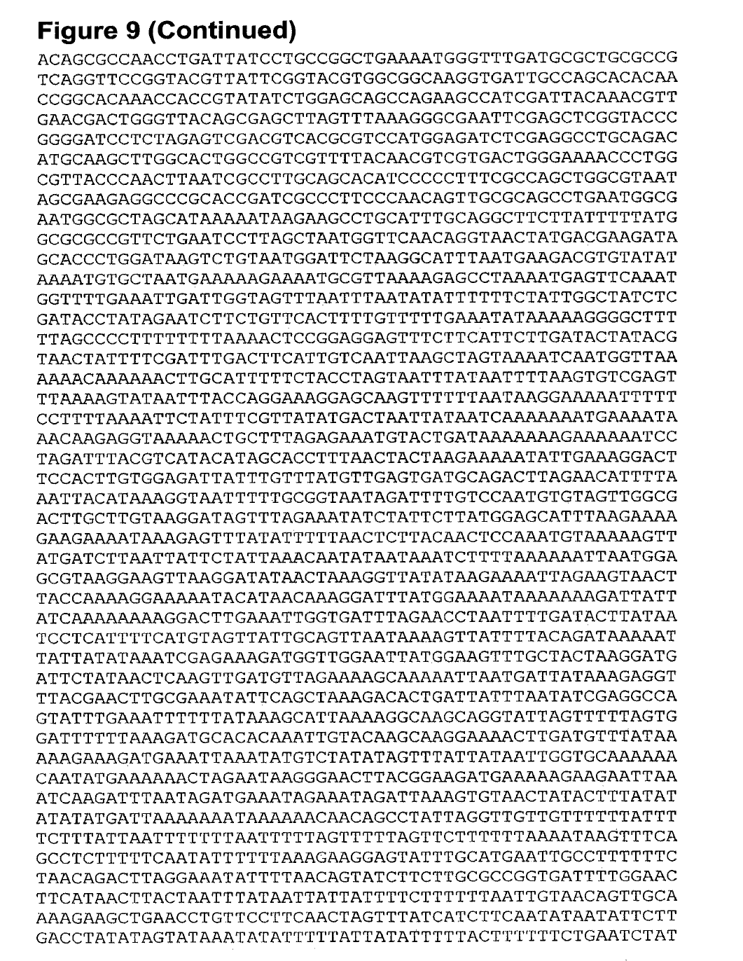

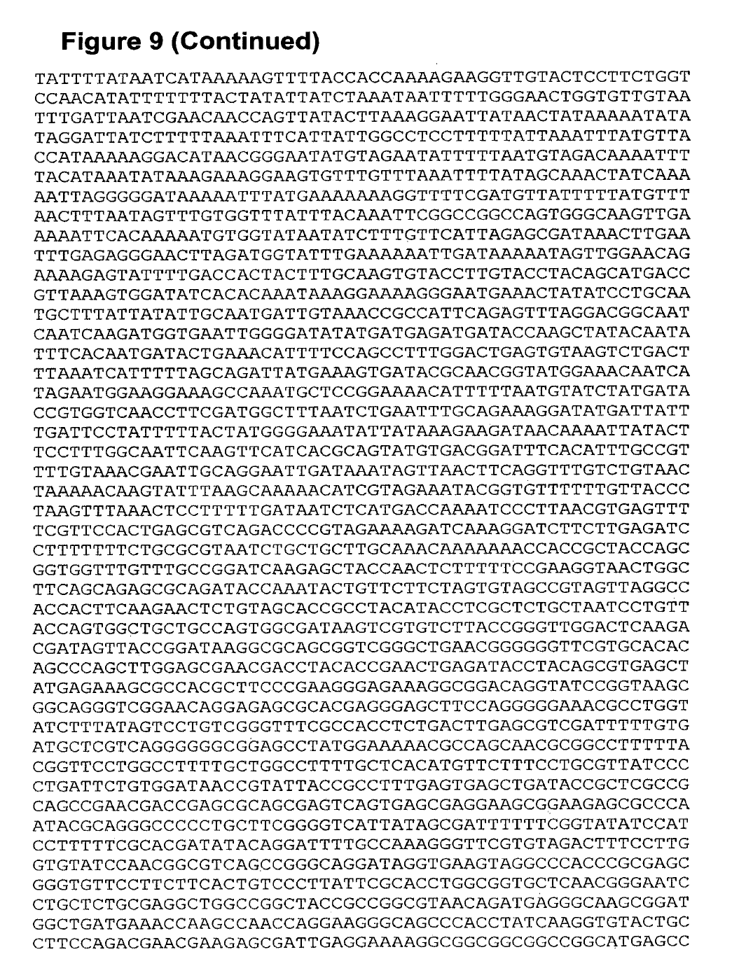

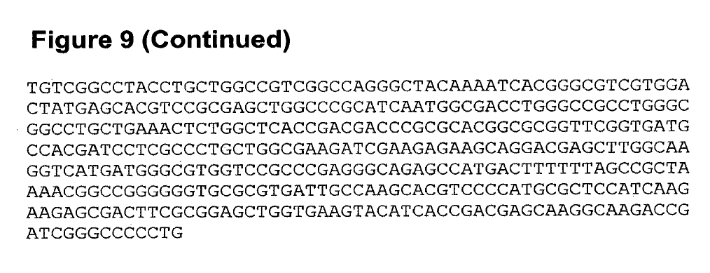

FIG. 9—codA allele exchange vector pMTL-SC7215 (SEQ ID NO: 2).

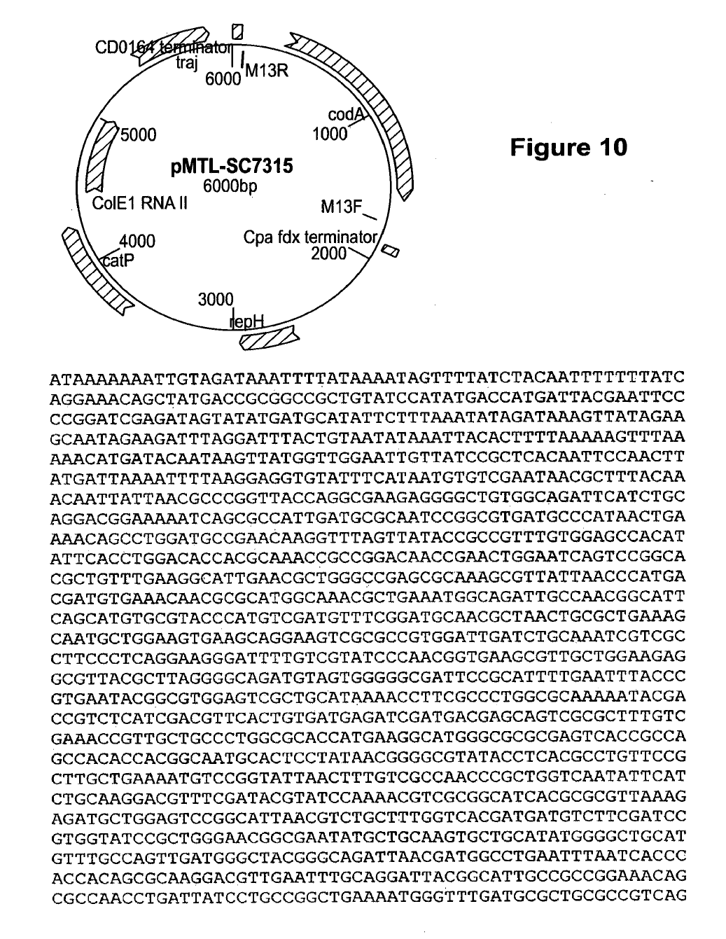

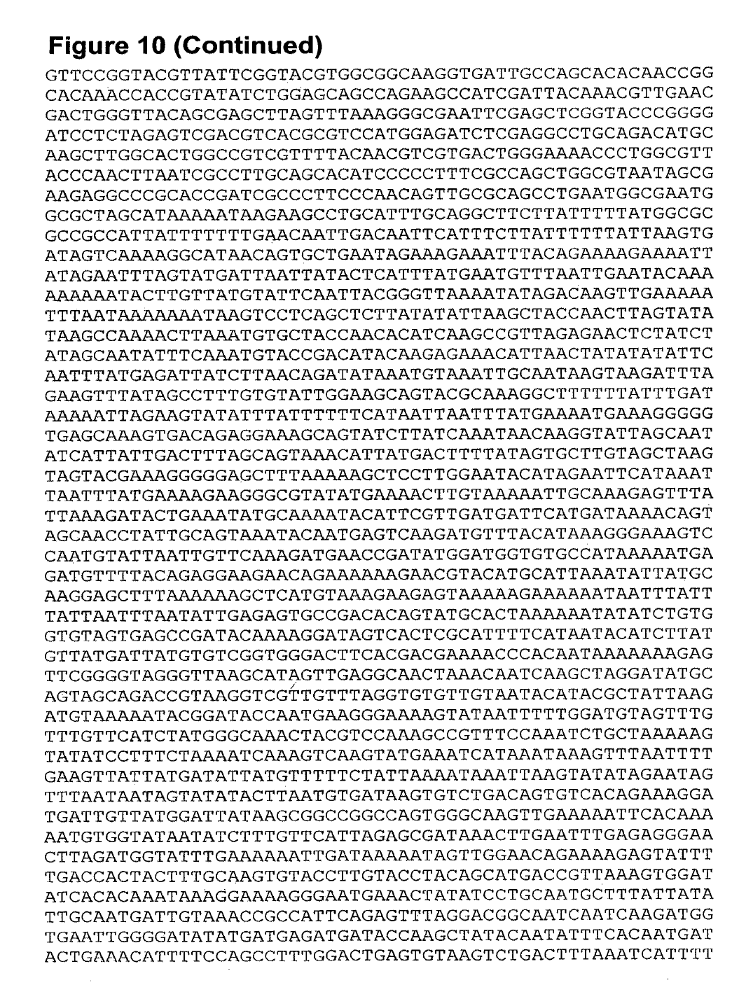

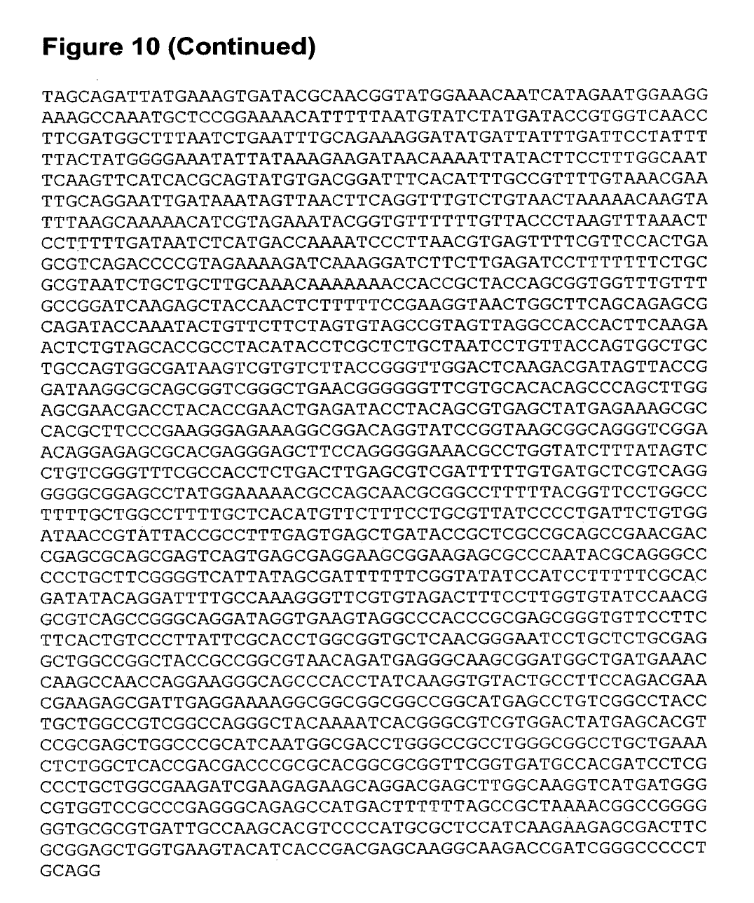

FIG. 10—codA allele exchange vector pMTL-SC7315 (SEQ ID NO: 3).

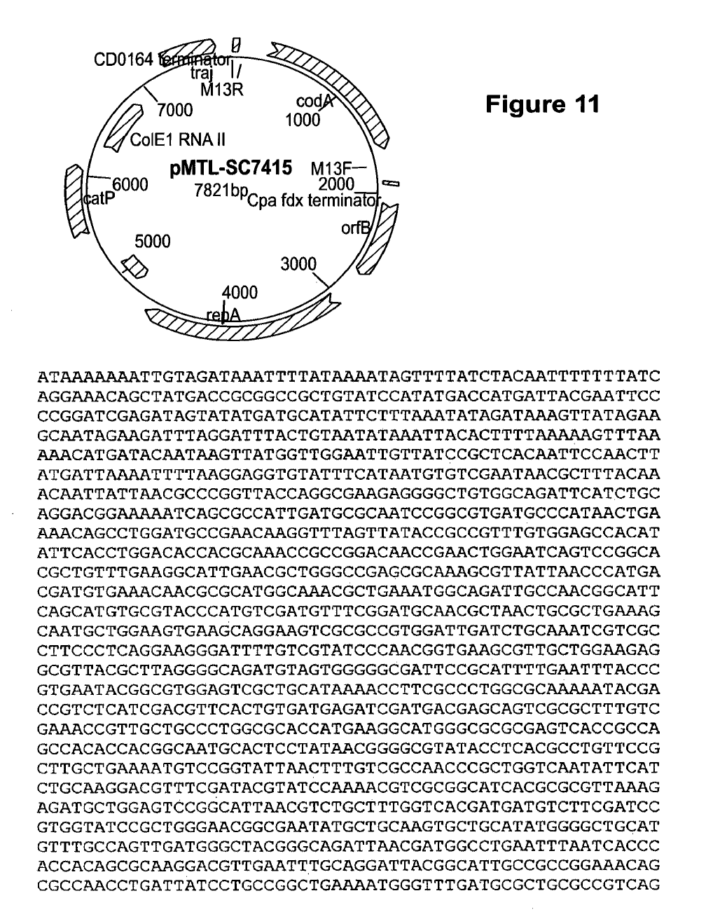

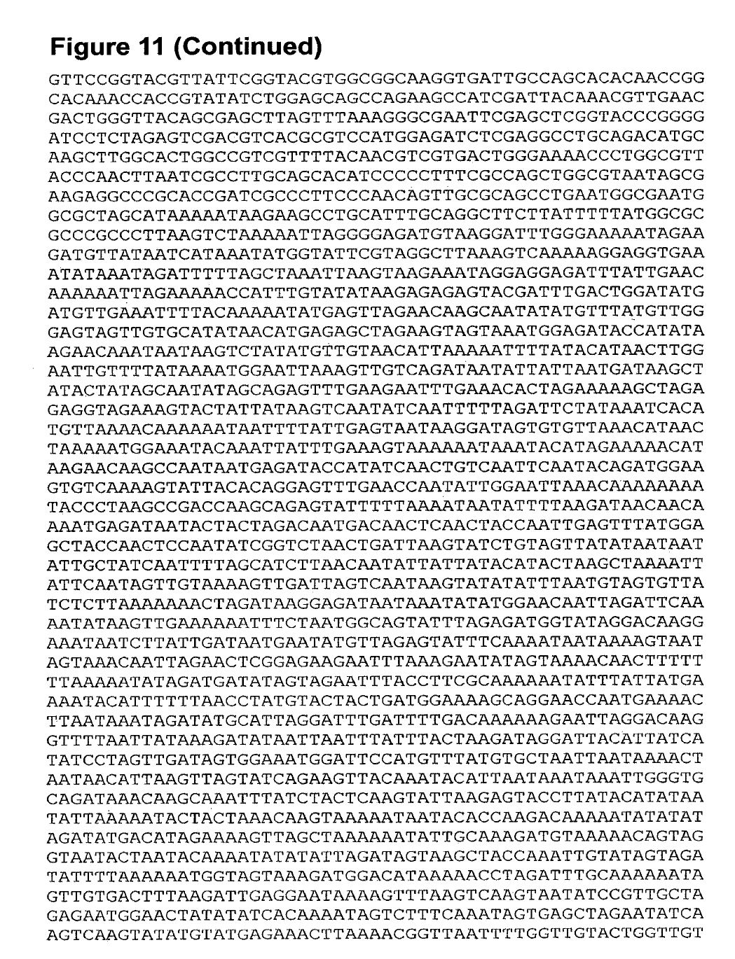

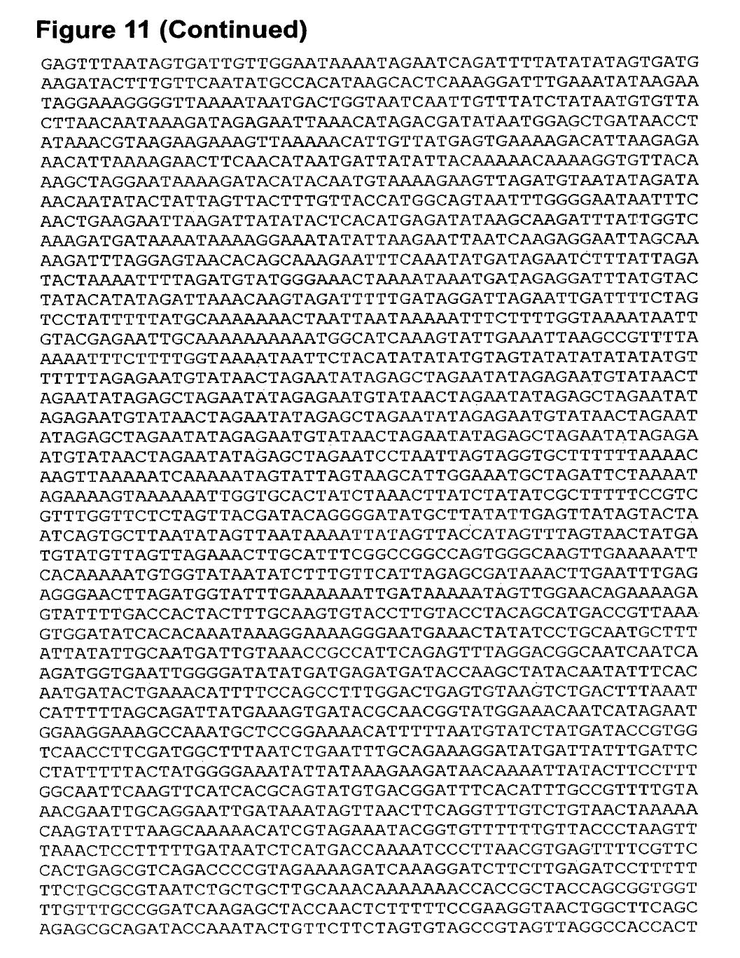

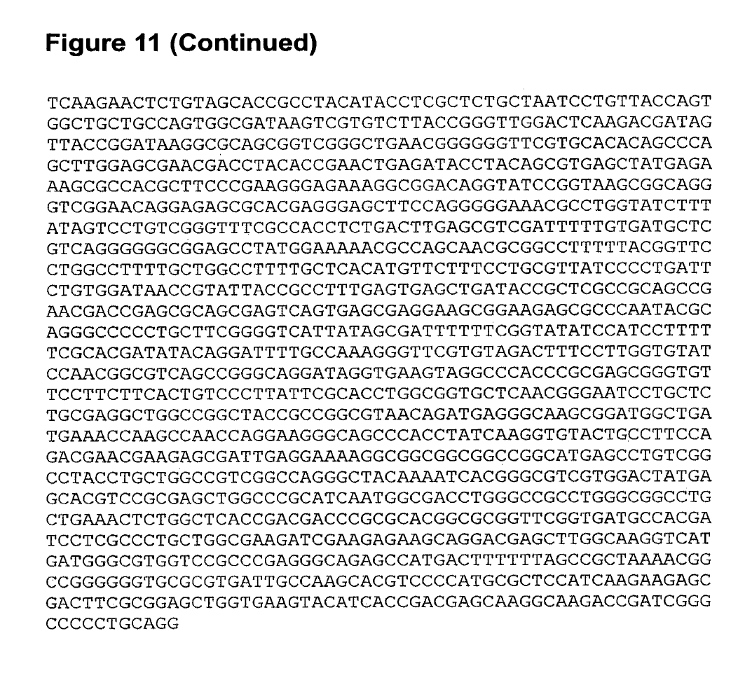

FIG. 11—codA allele exchange vector pMTL-SC7415 (SEQ ID NO: 4).

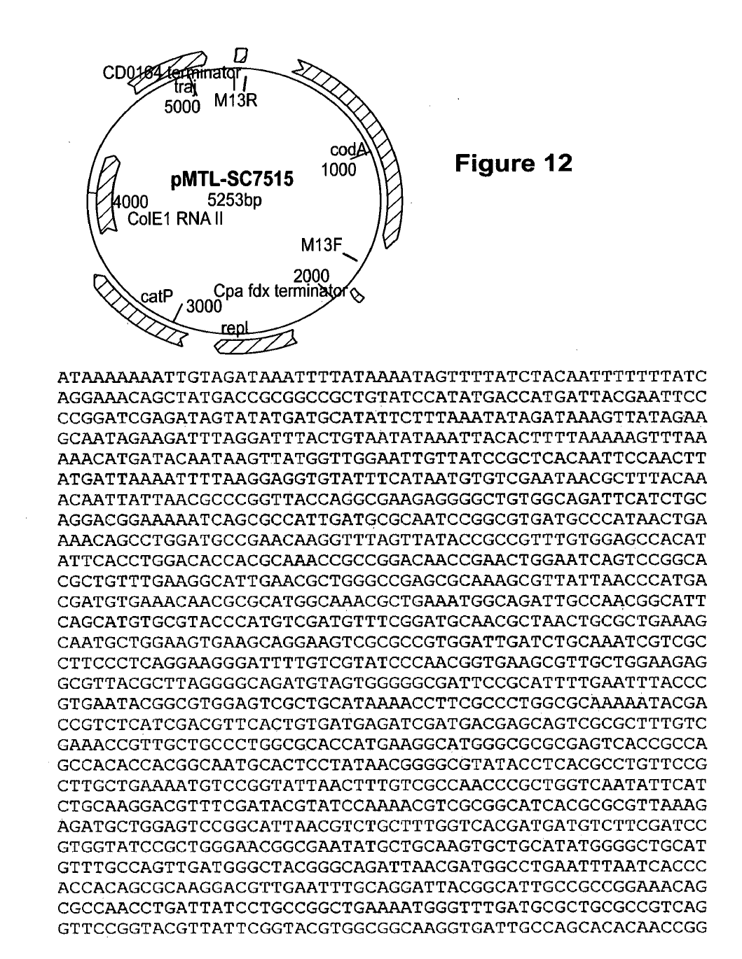

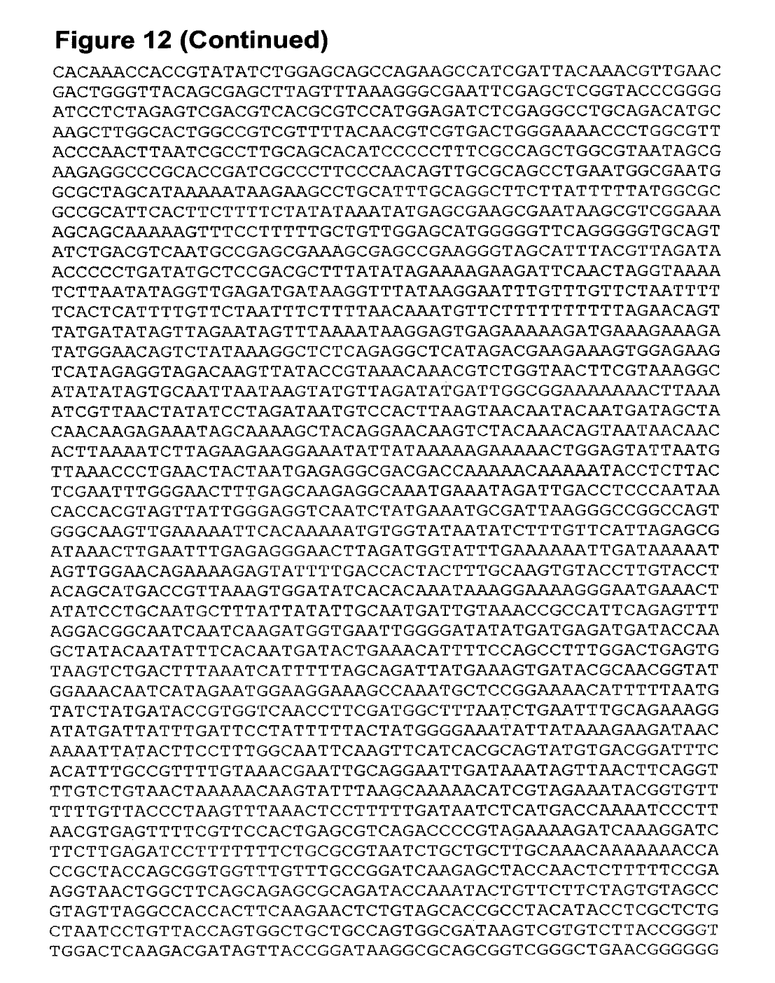

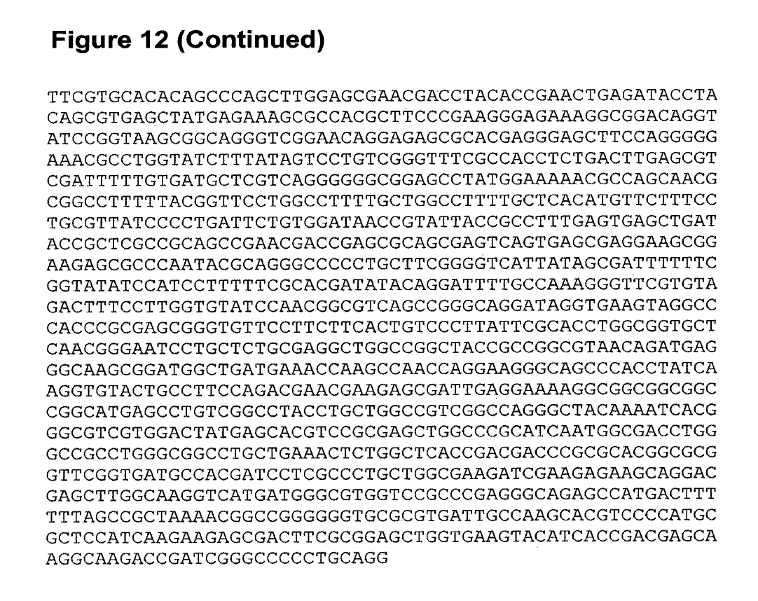

FIG. 12—codA allele exchange vector pMTL-SC7515 (SEQ ID NO: 5).

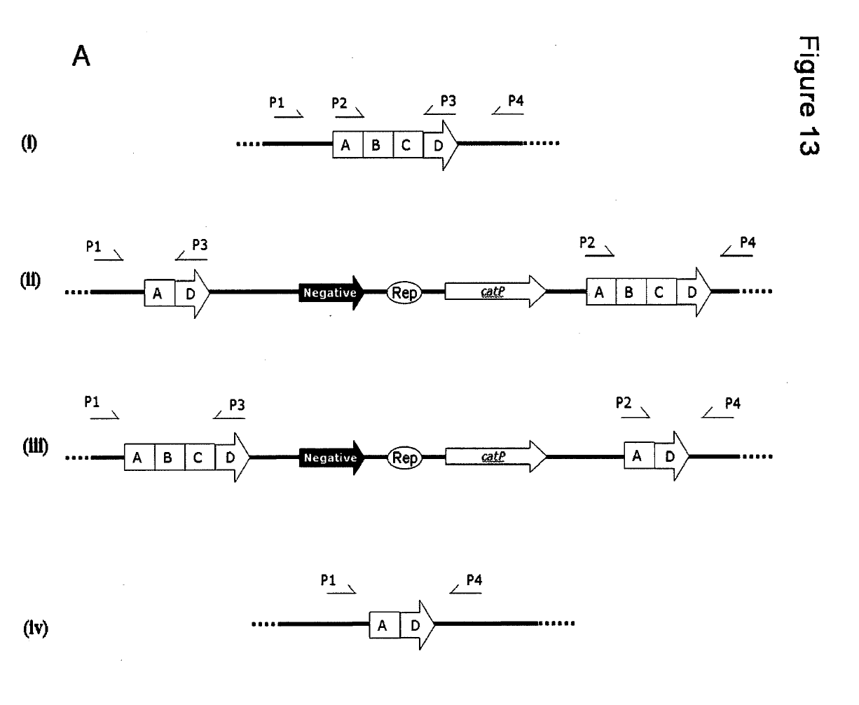

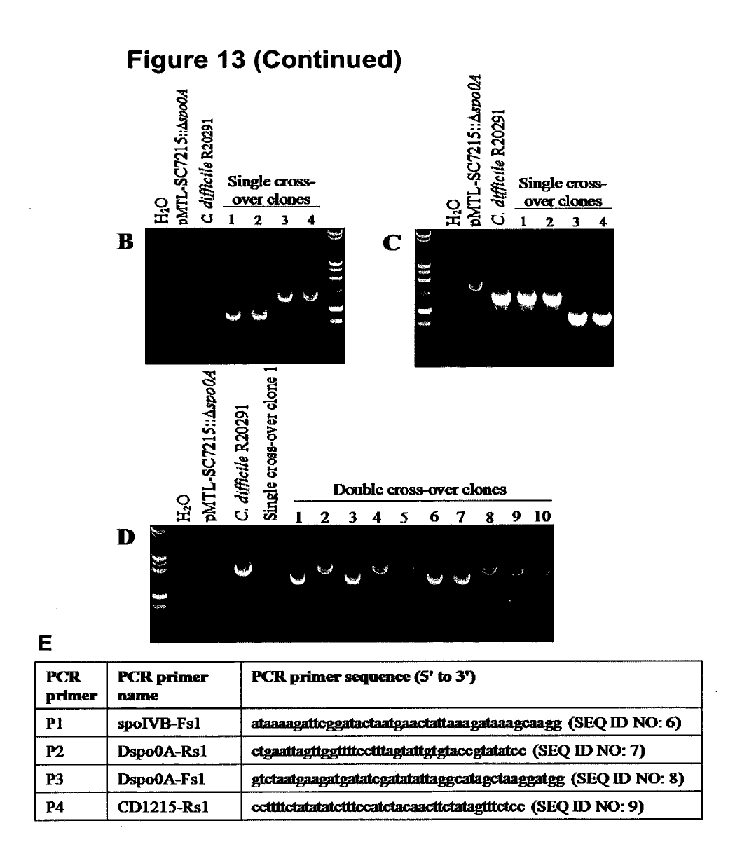

FIG. 13—Use of codA mediated allele exchange to construct a spo0A in-frame deletion mutant of C. difficile R20291. FIG. 13A—If the native chromosomal spo0A allele (i) is conceptually divided into the four segments A, B, C and D, the recombinant spo0A in-frame deletion allele (Δspo0A) consists of segments A and D only (iv). In this respect, segment A constitutes the LHA and segment D constitutes the RHA. Single cross-over clones were isolated following integration of pMTL-SC7215::Δspo0A into the chromosome, via a homologous recombination event in either the LHA or the RHA, as depicted in (ii) and (iii), respectively. Double cross-over clones were isolated following a second homologous recombination event, in the opposite homology arm to the first, and loss of the excised plasmid from the cell, which harboured the native chromosomal spo0A allele and the codA construct. This marked completion of the allele exchange process, whereby the native chromosomal spo0A allele had been exchanged for the recombinant spo0A in-frame deletion allele, Δspo0A. Confirmation of the process was gained by carrying out PCR's under conditions which favoured amplification of the smallest possible product. FIG. 13B—PCR with primers P1 and P3 proved the isolation of two single cross-over clones (clones 1 and 2), in which the first recombination event had occurred in the LHA. FIG. 13C—PCR with primers P2 and P4 proved the isolation of another two single cross-over clones (clones 3 and 4), in which the first recombination event had occurred in the RHA. FIG. 13D—Finally, PCR with primers P1 and P4 demonstrated the isolation of four double cross-over clones (clones 1, 3, 6, and 7) in which the native chromosomal spo0A allele had been exchanged for the smaller recombinant spo0A in-frame deletion allele, Δspo0A. These PCR products were sequenced for absolute confidence and were confirmed to be the recombinant spo0A in-frame deletion allele, Δspo0A. FIG. 13E-Details of screening primers used in exemplification of codA mediated allele exchange to construct a spo0A in-frame deletion mutant of C. difficile R20291.

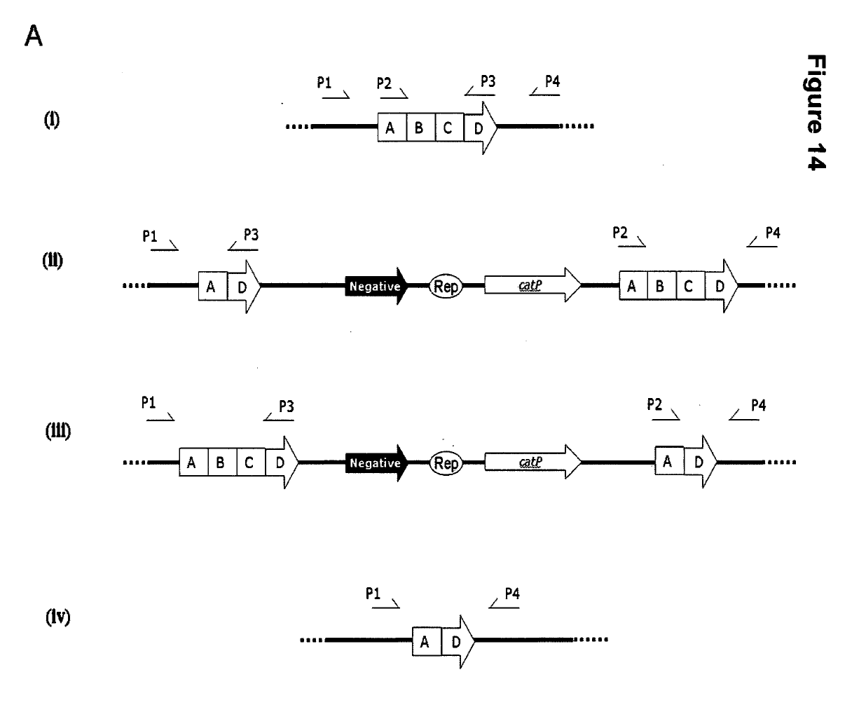

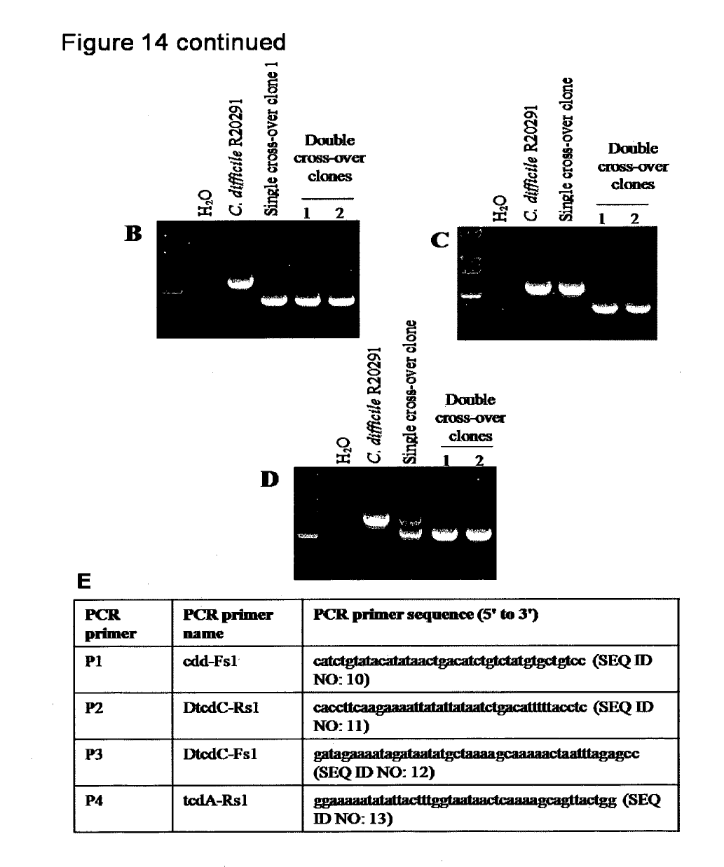

FIG. 14—Use of codA mediated allele exchange to construct a tcdC in-frame deletion mutant of C. difficile R20291. FIG. 14A-If the native chromosomal tcdC allele (i) is conceptually divided into the four segments A, B, C and D, the recombinant tcdC in-frame deletion allele (ΔtcdC) consists of segments A and D only (iv). In this respect, segment A constitutes the LHA and segment D constitutes the RHA. Single cross-over clones were isolated following integration of pMTL-SC7215::ΔtcdC into the chromosome, via a homologous recombination event in either the LHA or the RHA, as depicted in (ii) and respectively. Double cross-over clones were isolated following a second homologous recombination event, in the opposite homology arm to the first, and loss of the excised plasmid from the cell, which harboured the native chromosomal tcdC allele and the codA construct. This marked completion of the allele exchange process, whereby the native chromosomal tcdC allele had been exchanged for the recombinant tcdC in-frame deletion allele, ΔtcdC. FIGS. 14B and 14C—Confirmation of the process was gained by carrying out PCR's under conditions which favoured amplification of the smallest possible product. Separate PCR's carried out with primers P1 and P3 (B), and P2 and P4 (C), respectively, confirmed the isolation of a single cross-over clone in which the first recombination event had occurred in the LHA. FIG. 14D—PCR's carried out with primers P1 and P4 demonstrated the isolation of two double cross-over clones (clones 1 and 2) in which the native chromosomal tcdC allele had been exchanged for the smaller recombinant tcdC in-frame deletion allele, ΔtcdC (vii). These PCR products were sequenced for absolute confidence and were confirmed to be the recombinant spo0A in-frame deletion allele, ΔtcdC. FIG. 14E—Details of screening primers used in exemplification of codA mediated allele exchange to construct a tcdC in-frame deletion mutant of C. di/flak R20291.

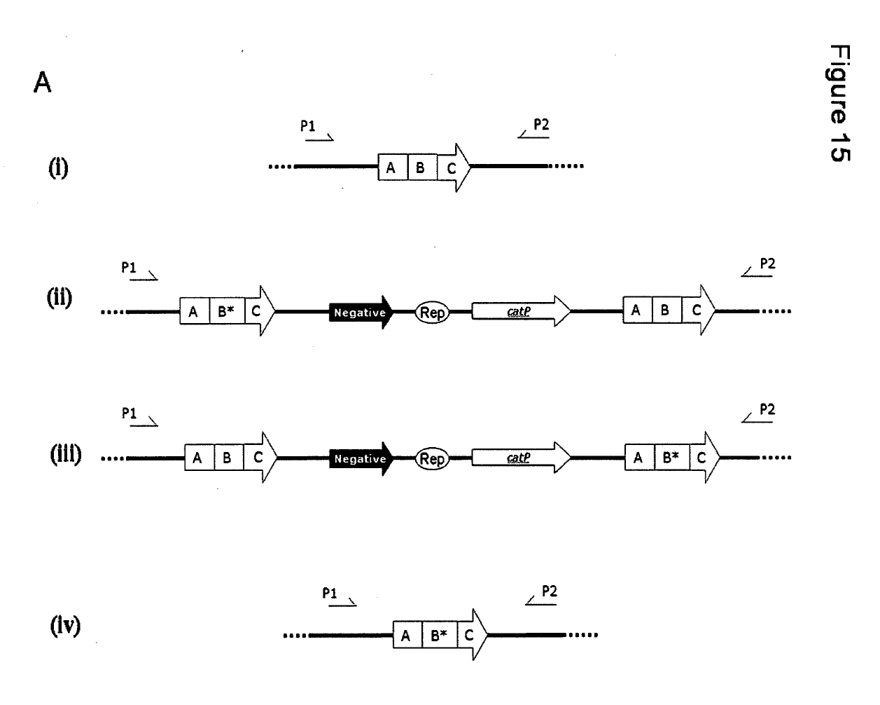

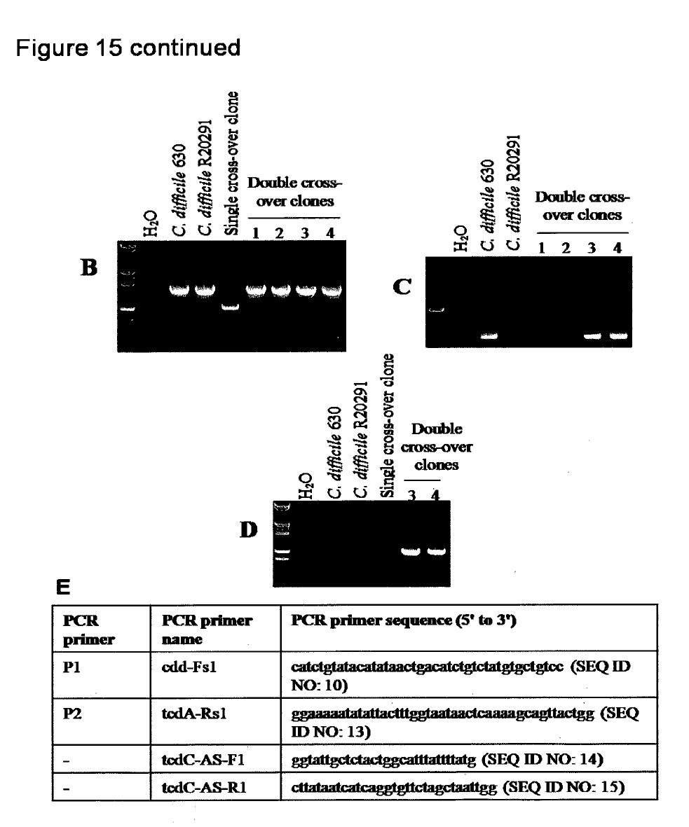

FIG. 15—Use of codA mediated allele exchange to insert a single base into the tcdC open-reading-frame of C. difficile R20291. FIG. 15A—If the native chromosomal tcdC allele (i) is conceptually divided into three segments A, B and C, the recombinant tcdC::117A allele (iv) consists of segments A, B* and C, where B* differs from B only by one additional base-pair (ie. 117A). In this respect, segment ‘A’ constitutes the LHA and segment ‘C’ constitutes the RHA. Single cross-over clones were isolated following integration of pMTL-SC7215:: tcdC::117A into the chromosome, via a homologous recombination event in either the LHA or the RHA, as depicted in (ii) and (iii), respectively. Double cross-over clones were isolated following a second homologous recombination event, in the opposite homology arm to the first, and loss of the excised plasmid from the cell, which harboured the native chromosomal tcdC allele and the codA construct. This marked completion of the allele exchange process, whereby the native chromosomal tcdC allele had been exchanged for the recombinant tcdC::117A allele. FIGS. 15B and 15C—Confirmation of the process was gained by carrying out PCR with primers P1 and P2, which clearly demonstrated that four double cross-over clones had been isolated from single cross-over clone 1. Sequencing the PCR products arising from each of the four double cross-over clones revealed that clones 3 and 4 were recombinants which harboured the tcdC::117A allele in the chromosome in place of the R20291 wild-type tcdC allele. Clones 1 and 2 were wild-type revertants which still harboured the R20291 wild-type tcdC allele, due to the second homologous recombination event occurring in the same homology arm as the first. These results were confirmed by allele-specific PCR using primers ‘tcdC-AS-F1’ and ‘tcdC-AS-R1’, which only yield a PCR product if the template DNA harbours the tcdC::117A allele (C). FIG. 15D—Finally, to confirm that allele-specific PCR products were not the result of contaminating C. difficile 630 positive control DNA, allele-specific PCR's were repeated using primers ‘tcdC-AS-F1’ and ‘tcdA-Rs1’ which would only yield a PCR product from C. difficile R20291DNA harbouring the tcdC::117A allele. FIG. 15E—Details of screening primers used in exemplification of codA mediated allele exchange to insert a single base into the tcdC open-reading-frame of C. difficile R20291.

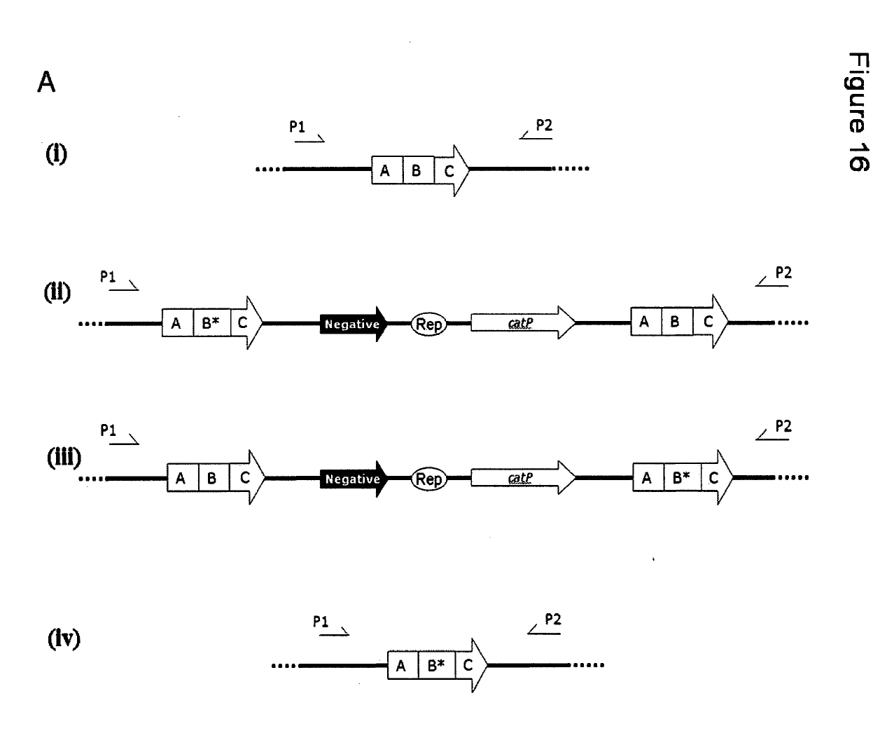

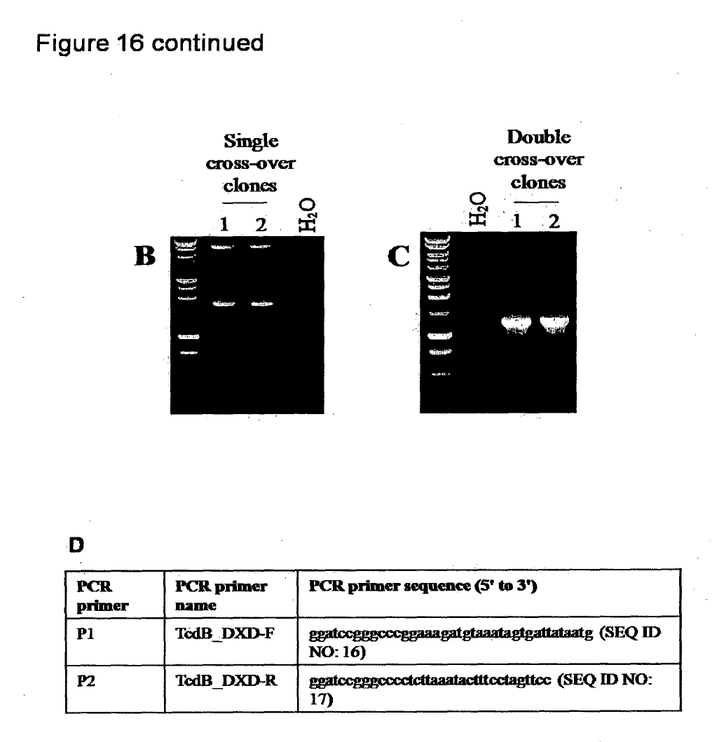

FIG. 16—Use of codA mediated allele exchange to alter the catalytic ‘DXD’ domain of tcdB to ‘AXA’. FIG. 16A—The native chromosomal tcdB allele (i) is conceptually divided into three segments A, B and C, where ‘B’ has the DNA sequence ‘ATGTTGA’. The recombinant tcdB-DXD286/8AXA allele (iv) consists of segments A, B* and C, where segment ‘A’ constitutes the LHA, segment ‘C’ constitutes the RHA, and ‘13*’ differs from ‘B’ in that it has the DNA sequence CTGTTGC. Single cross-over clones were isolated following integration of pMTL-SC7315:: tcdB-DXD286/8AXA into the chromosome, via a homologous recombination event in either the LHA or the RHA, as depicted in (ii) and (iii), respectively. Double cross-over clones were isolated following a second homologous recombination event, in the opposite homology arm to the first, and loss of the excised plasmid from the cell, which harboured the native chromosomal tcdB allele and the codA construct. This marked completion of the allele exchange process, whereby the native chromosomal tcdB allele had been exchanged for the recombinant tcdB-DXD286/8AXA allele. FIGS. 16B and 16C—Confirmation of the process was gained by carrying out PCR with primers P1 and P2, which clearly demonstrated that two single cross-over clones were isolated (B) and two double cross-over clones were isolated (C). Sequencing of the PCR products arising from each of the two double cross-over clones revealed that both were recombinants which harboured the recombinant tcdB-DXD286/8AXA allele on the chromosome in place of the R20291 wild-type tcdB allele. FIG. 16D—Details of screening primers used in exemplification of codA mediated allele exchange to alter the catalytic ‘DXD’ domain of tcdB to ‘AXA’.

### CONSTRUCTION OF CODA EXPRESSING PLASMID

The codA expression cassette was isolated from the vector used by Fox et al., Gene Therapy. (1996) 3:173-178 and cloned into pMTL960. This plasmid was used to test the functionality of the codA gene in E. coli and C. difficile. In E. coli the construct functioned as expected, permitting growth in the absence of FC but not in the presence of FC. Similarly, as illustrated in FIG. 4, when transformed into C. difficile and grown on minimal medium (modified from a recipe described by Karlsson et al. (1999) Microbiology 145:1683-1693) containing 100 μg/ml FC, C. difficile cells harbouring the codA cassette could be differentiated. That is, cells expressing codA did not grow.

The medium described by Karlson et al comprised:

| | | Minerals, | |
|---|---|---|---|
| | Concentration | carbohydrates | Concentration |
| Amino acids | (mg/ml) | and vitamins | (mg/ml) |
| Tryptophan (W)* | 0.1 | CoCl₂•6H₂O | 0.001 |
| Methionine (M)* | 0.2 | FeSO₄•7H₂O | 0.004 |
| Isoleucine (I)* | 0.3 | MnCl₂•4H₂O | 0.01 |
| Proline (P)* | 0.3 | MgCl₂•6H₂O | 0.02 |
| Valine (V)* | 0.3 | CaCl₂•2H₂O | 0.026 |
| Leucine (L)* | 0.4 | (NH4)₂SO₄ | 0.04 |
| Cysteine (C)* | 0.5 | KH₂PO₄ | 0.9 |
| Glycine (G)** | 0.1 | NaCl | 0.9 |
| Threonine (T)** | 0.2 | NaHCO₃ | 5 |
| Histidine (H)*** | 0.1 | Na₂HPO₄ | 5 |
| Tyrosine (Y)*** | 0.1 | | |
| Alanine (A)*** | 0.2 | Glucose | 2 |
| Arginine (R)*** | 0.2 | | |
| Phenylalinine | 0.2 | D-Biotin | 0.000012 |
| (F)*** | | | |
| Aspartic acid | 0.4 | Calcium D- | 0.001 |
| (D)*** | | pantothenate | |
| Lysine (K)*** | 0.4 | Pyridoxine | 0.001 |
| Serine (S)*** | 0.4 | | |

*Amino acids in minimal defined medium (MDM)
**Further amino acids added to give supplemented defined medium (SDM)
***Further amino acids added to give complete defined medium (CDM)

The modified media used in this example comprised:

| | | Minerals, | |
|---|---|---|---|
| | Concentration | carbohydrates | Concentration |
| Amino acids | (mg/ml) | and vitamins | (mg/ml) |
| Cas-aminoacids | 100 | CoCl₂•6H₂O | 0.001 |
| Tryptophan (W) | 0.5 | FeSO₄•7H₂O | 0.004 |
| Cysteine (C) | 0.5 | MnCl₂•4H₂O | 0.01 |
| | | MgCl₂•6H₂O | 0.02 |
| | | CaCl₂•2H₂O | 0.026 |
| | | (NH4)₂SO₄ | 0.04 |
| | | KH₂PO₄ | 0.9 |
| | | NaCl | 0.9 |
| | | NaHCO₃ | 5 |
| | | Na₂HPO₄ | 5 |
| | | Glucose | 10 |
| | | D-Biotin | 0.001 |
| | | Calcium D- | 0.001 |
| | | pantothenate | |
| | | Pyridoxine | 0.001 |

Exemplification of codA as a Negative Selection Marker/Use of codA to Knockout the spo0A Gene of C. difficile

Having demonstrated that it is possible to select against C. difficile cells harbouring the codA cassette on minimal medium supplemented with FC, a stable spore minus mutant of C. difficile 027 (R20291, isolated from the Stoke Mandeville UK outbreak) was constructed using the codA cassette as a negative selection marker.

The shuttle vector depicted in FIG. 5 was constructed. This vector harboured both the codA negative selection marker and a spo0A knock-out cassette (ie. the spo0A gene of C. difficile interrupted by the catP gene which confers resistance to chloramphenicol and thiamphenicol). The catP serves as the selectable marker to screen for first recombination event products.

To isolate the single cross-over mutant/first recombination event product, the vector was transferred into C. difficile and transconjugant colonies with an apparent growth advantage (ie. those with a visibly faster growth rate) under thiamphenicol selection were selected.

Products of the first recombination were screened by PCR, and it is clear from the results in FIG. 6 that single cross-over integrants in which homologous recombination had occurred in either the left or the right homology arm of the spo0A knock-out cassette had been isolated.

The single cross-over integrants were then serially passaged in minimal medium (ie. with no exogenous pyrimidines) and plated onto medium supplemented with 50 μg/ml FC. Of the individual colonies isolated, seven were screened by PCR to see if any were double crossover spo0A mutants which had lost the plasmid (and hence had become resistant to FC). Of the seven clones screened, two appeared to be spontaneous mutants resistant to FC while the remaining five had arisen through double crossover excision of the plasmid. In two cases, the excision event had resulted in the desired mutant, while in the other 3 cases the wild-type allele had been re-created in the chromosome, generating wild-type strains (FIG. 7).

The results show for the first time an example of a negative selection marker which may be deployed to generate double crossover mutants in Clostridia.

This method has the advantage that no unwanted exogenous DNA is left behind from the donor DNA molecule in the method, the only DNA retained is the exchanged DNA which it is desired to be retained.

The negative selection marker codA may be used to create ‘perfect’ in frame deletions, where the target gene can be precisely deleted with no effect on up or downstream genes. It can also be used to introduce larger DNA fragments than the ClosTron (Heap et al. (2007) Journal of Microbiological Methods 70:452-464) encoding desired advantageous properties, eg., plant degrading enzymic activities or therapeutic anti-cancer agents useful in the CDEPT strategy. Moreover, it may also be used to substitute specific wild type genes in the chromosome with rationally altered alleles. For example, it could be used to introduce gene variants carrying specific base pair deletions or substitutions, such as a copy of a toxin gene encoding a rationally altered product lacking toxicity. This approach could be used to generate a strain of C. di/fiche producing an inactive toxin that would not require formalin treatment to produce a vaccine candidate.

Standardised Protocol for codA Mediated Allele Exchange in Clostridium spp.

The procedure for codA mediated allele-exchange in Clostridium spp. has been standardised into the following protocol:

1. Construct recombinant allele by PCR or DNA synthesis.
2. Clone recombinant allele into replication-defective codA allele exchange vector. Preferably, pMTL-SC7215 (FIG. 9), pMTL-SC7315 (FIG. 10), pMTL-SC7415 (FIG. 11) or pMTL-SC7515 (FIG. 12).
3. Confirm sequence of recombinant allele via DNA sequencing.

Preferably, using primers

SC7-Fs1
(SEQ ID NO: 18))
(5′-GACGGATTTCACATTTGCCGTTTTGTAAACGAATTGCAGG-3′
and/or
SC7-Rs1
(SEQ ID NO: 19))
(5′-AGATCCTTTGATCTTTTCTACGGGGTCTGACGCTCAGTGG-3′.

4. Transfer codA allele-exchange vector harbouring recombinant allele into cells of Clostridium spp. via transformation or conjugation. Select for transformants/transconjugants using and appropriate growth medium supplemented with the appropriate antibiotic (ie. the antibiotic for which resistance is encoded in codA allele exchange vector).
5. Obtain single cross-over mutant/first recombination event product, by re-streaking transformant/transconjugant onto fresh medium of the same composition and isolating colonies with a growth advantage under antibiotic selection (ie. those with a visibly faster growth rate). Single cross-over integrants can be confirmed by PCR at this stage.
6. To isolate double cross-over mutants, the pure single cross-over integrant culture the pure single cross-over integrant in the absence of selection, preferably between 48 and 120 hours. Harvest all growth into phosphate buffered saline (PBS) and plate serial dilutions onto medium supplemented with FC, preferably minimal medium supplemented with 50 μg/ml FC.
7. Patch plate colonies which are visible after 24 to 48 hours of incubation onto the same medium, supplemented with FC, and separately onto growth medium supplemented with the antibiotic for which resistance was encoded on the codA allele exchange vector used.
8. Colonies which grow on medium supplemented with FC, but not on medium supplemented with antibiotic are likely to be double cross-over integrants. Confirm these by PCR and/or sequencing.
Further Exemplification of Using codA a Negative Selection Marker for Precise Manipulation of the Clostridium difficile Genome.

Earlier, an example of codA mediated allele-exchange was described in which recombinant strain C. difficile R20291 spo0A::catP was constructed (i.e. the spo0A gene of C. difficile R20291 was interrupted by the antibiotic resistance gene catP, thus rendering the strain chloramphenicol/thiamphenicol resistant and unable to for spores).

The codA mediated allele exchange has been exemplified a further four times. On each occasion the ‘standardised protocol’ detailed above was used to isolate the recombinant Clostridium strain. Each of these exemplifications of the technology is described in turn in the following text:

1) Use of codA Mediated Allele Exchange to Construct a spo0A in-Frame Deletion Mutant of C. difficile R20291.

The recombinant spo0A in-frame deletion allele was constructed by splicing-by-overlap (SOE) PCR using the following primers:

Dspo0A-LHA-F3:
(SEQ ID NO: 20)
ttttttGACGTCggtaaaataaaaggagattttaatgacagcaatttaa
tggg (53)
Dspo0A-LHA-R1:
(SEQ ID NO: 21)
ccatgcaacctccattattacatctagtattaataagtccggttgtg
(47)
Dspo0A-RHA-F1:
(SEQ ID NO: 22)
gatgtaataatggaggttgcatggagtagaggaaaagttgacac (44)
Dspo0A-RHA-R3:
(SEQ ID NO: 23)
ttttttGACGTCctccaacattatcaattattagtatattattttcagt
taatatccc (58)

This yielded the spo0A in-frame deletion construct with the following sequence (SEQ ID NO: 24):

ttttttGACGTCGGTAAAATAAAAGGAGATTTTAATGACAGCAATTTA
ATGGGTAATTTCTCAAATAATTCAGAGCTAGGTATAAGTGGTAAT
ATTACAGAAAACCATAATAAAGAGTTTAATGTAGCAAATAAAGAA
AAGCAATTAATAGAAGTTGGAAGGCCGCAAGATGTAAAAATAGGA
GATGCAGTAATTCTTTTTGAGGATAAAAACAAAAATATAACAAGC
TATGATATAAAAATAGAAAGTATAGTATATGATAAAGGAAATTAT
AGAGATATGGTAATAAAAGTAGTAGATGACAAGTTATTGGAATAC
ACAGGAGGTATCGTACAGGGGATGAGTGGAGCTCCAATAATACAA
AATAATAAAATTATTGGTGCAATAACTCATGTTTTTAGAGATAATC
CGAAAAAAGGATATGGTATTTTTATAGATGAAATGATAAAATTGT
AGGTGAGGCATTAAAAAATTTTATTATTTTATCAATTATCTAGGAG
GAATATAATTTTGGAGTGTCGAATATGCTTTAGAGTAGATAATTAG
GAAGCAATTGTGTAAAAAGTTTAGTTTTCTGTAATAAGAAGATGTT
TTTTAATGGGGGGATTTTTAGTGGAAAAAATCAAAATAGTTTTAGC
AGATGACAATAAGGATTTTTGTCAGGTATTAAAAGAGTATTTGTCT
AATGAAGATGATATCGATATATTAGGCATAGCTAAGGATGGAATT
GAAGCATTAGACTTAGTAAAAAAGACACAACCGGACTTATTAATA
CTAGATGTAATAATG*GAGGTTGCATGGAGTAGAGGAAAAGTTGA
CACAATAAATCAATTATTTGGATATACGGTACACAATACTAAAGG
AAAACCAACTAATTCAGAATTTATAGCAATGATTGCTGATAAATT
AAGACTAGAACATAGTATGGTTAAATAAACAAGACATAAAAAGTA
AGGCTTTTTAATTAAGGCATTGGCTATAAATGCGTATTACAAGCAG
CGAAACGGTATAACCACTAGGGTTATACCGTTTCGCTATTTTAATA
AATATAAAAATTTTCTTTATTATTTGCTTACTATATCAATATAATA
ATTTTATTATACTATGGATATAGTATGTGTCTTTACAAGTTGTAAA
CTGACAGTGGTTTATTTTTTAATATAAATATTGACTTTGATGCAGG
TAAACTTTGTATTTTTAAGCGTATTGTGGAATATGTTAAATAAAAA
AATGATGAAATATAGTATTGTAAATGCCAAAGATGCAAAACAAAC
TTAAAACATTTATTTTATTGTTAAGTAATGCTATAATATAATGTGA
TTTTAATAATGATAGTGGAGGTTTAAATATGAGAGTCGAGGCCCC
TATAAAAGTAGATCGAAAAACCAAAAAACTTGCTAAAAGAGTTGA
AAGTGGGGAAATAGCAGTTATAAATCATATAGACATAGATGAAGT
TGCTGCAAACTCTTTAGTAGAAGCTAAAATAAAACTTGTCATAAA
TGCGGCTCCTTCTATAAGTGGTAGGTATCCCAATAAAGGTCCAGG
GATATTAACTGAAAATAATATACTAATAATTGATAATGTTGGAGG
ACGTCaaaaaa

This construct has a left-homology-arm (LHA) of 777 bp and a right-homology-arm (RHA) of 800 bp (separated by a ‘*’ in the sequence shown above). It specifies a 486 bp deletion in the spo0A open-reading-frame (ORF), which is 825 bp in total. This constitutes deletion of codons 65 to 226 inclusive, out of a total of 275 codons, and renders the spo0A gene-product of C. difficile R202911n-active.

The recombinant spo0A in-frame deletion allele was cloned as a ZraI fragment into the Pinel site of pMTL-SC7215 (FIG. 9), to give pMTL-SC7215::Δspo0A. This vector was transferred into C. difficile R20291 by conjugation from E. coli CA434. Single and double cross-over clones were then isolated sequentially using the standardised protocol for codA mediated allele exchange as described above. Single and double cross-over clones were confirmed by PCR and sequencing (FIG. 13).

2) Use of codA Mediated Allele Exchange to Construct a tcdC in-Frame Deletion Mutant of C. difficile R20291.

The recombinant tcdC in-frame deletion allele was constructed by SOE-PCR using the following primers:

DtcdC-027-LHA-F1:
(SEQ ID NO: 25)
ttttttGACGTCtttccctacccctggaattttttgtagttctcccata
cttcacc (56)
DtcdC-027-LHA-R1:
(SEQ ID NO: 26)
cagctatccccttagagcttccttttctttcattactaaattcgttacc
(49)
DtcdC-027-RHA-F1:
(SEQ ID NO: 27)
ggaagctctaaggggatagctgtagagaaaattaattaatattgttttg
(49)
DtcdC-027-RHA-R1:
(SEQ ID NO: 28)
ttttttGACGTCgtatattactttatgcctgatactgctatggctgcag
ctggtgg (56)

This yielded the tcdC in-frame deletion construct with the following sequence (SEQ ID NO: 29):

ttttttGACGTCTTTCCCTACCCCTGGAATTTTTTGTAGTTCTCCCATA
CTTCACCTTCTTTCTGATATATTATTTTTGTATTATACTTAGTACCAG
ATATTTTTTATTATAGTTAATATTTAATTTTTATTATATCACTTTAT
TTATGCTCTTTCATCTATCTATATTTTACCACCTCTAAAGTACTGA
ATCATTTAATTACATCATAATATAGTTTTATACAAATAAAATACTT
TATGTTTCATTTAATATATAAAATTCACCTTCAAGAAAATTATATT
ATAATCTGACATTTTTACCTCATTTTTCAAAATATATTGAATCTTC
TTGATTTATTTTGTAAAATTATGCTTAGGGGAAATATATTTTAGGA
AAATATGAATATATAATTTTTAGTCAACTAGTTATTTTAAGTTTTT
AAATTTTAAAATAAAATATATCTAATAAAAGGGAGATTGTATTAT
GTTTTCTAAAAAAAATGAGGGTAACGAATTTAGTAATGAAAGAAA
AGGAAGCTCTAAG*GGGATAGCTGTAGAGAAAATTAATTAATATT
GTTTTGTATTATAGTTAATATTTTATATTATAGTCAATATGTTTAA
AGATGTTTTTATAATTGCAAATAAACAGTTACAAGGCTCTAAATTA
GTTTTTGCTTTTAGCATATTATCTATTTTCTATCAACTATTAATTAT
TTAGTATTAATATTTCCATATATGAATTTTATTATAAAATAGTCAA
GAATAATAGATTATTAAATGATAGAAAAATTTTAACTAAAAGTCA
TGTATTACAATAACACATGACTTTTAATTAAATCTCAATATTTATT
ATATAAAAATAATTTCTGAGTATCACAGGAATAATTTTTTGTCAAA
CATATATTTTAGCCATATATCCCAGGGGCTTTTACTCCATCAACAC
CAAAGAAATATATAACACCATCAATCTCGAAAAGTCCACCAGCTG
CAGCCATAGCAGTATCAGGCATAAAGTAATATACGACGTCaaaaaa

This construct has a LHA of 511 bp and a RHA of 478 bp (separated by a ‘*’ in the sequence shown above). It specifies a 593 bp deletion in the tcdC ORF, which is 677 bp in total.

The recombinant tcdC in-frame deletion allele was cloned as a ZraI fragment into the PmeI site of pMTL-SC7215 (FIG. 9), to give pMTL-SC7215::ΔtcdC. This vector was transferred into C. difficile R20291 bp conjugation from E. coli CA434. Single and double cross-over clones were then isolated sequentially using the standardised protocol for codA mediated allele exchange as described above. Single and double cross-over clones were confirmed by PCR and sequencing (FIG. 14).

3) Use of codA Mediated Allele Exchange to Insert a Single Base into the tcdC Open-Reading-Frame of C. difficile R20291.

The recombinant tcdC allele with a single dATP inserted at position 117 (ie. tcdC::117A) was constructed by SOE-PCR using the following primers:

117A-027-LHA-F1:
(SEQ ID NO: 30)
ttttttGACGTCtttccctacccctggaattttttgtagttctcccata
cttcacc (56)
117A-027-RHA-R1:
(SEQ ID NO: 31)
cacaccTaaaataaatgccagtagagcaatatcctttgtgctc (43)
117A-027-RHA-F1:
(SEQ ID NO: 32)
ggcatttattttAggtgtgttttttggcaatatatcctcaccagc (45)
117A-027-RHA-R1:
(SEQ ID NO: 33)
ttttttGACGTCtttctctacagctatccctggtatggttatttttcca
ccc (52)

This yielded the tcdC::117A construct with the following sequence (SEQ ID NO: 34):

ttttttGACGTCTTTCCCTACCCCTGGAATTTTTTGTAGTTCTCCCATAC
TTCACCTTCTTTCTGATATATTATTTTTGTATTATACTTAGTACCAG
ATATTTTTTATTATAGTTAATATTTAATTTTTATTATATCACTTTAT
TTATGCTCTTTCATCTATCTATATTTTACCACCTCTAAAGTACTGA
ATCATTTAATTACATCATAATATAGTTTTATACAAATAAAATACTT
TATGTTTCATTTAATATATAAAATTCACCTTCAAGAAAATTATATT
ATAATCTGACATTTTTACCTCATTTTTCAAAATATATTGAATCTTC
TTGATTTATTTTGTAAAATTATGCTTAGGGGAAATATATTTTAGGA
AAATATGAATATATAATTTTTAGTCAACTAGTTATTTTAAGTTTTT
AAATTTTAAAATAAAATATATCTAATAAAAGGGAGATTGTATTAT
GTTTTCTAAAAAAAATGAGGGTAACGAATTTAGTAATGAAAGAAA
AGGAAGCTCTAAGAAAATAATTAAATTCTTTAAGAGCACAAAGGA
TATTGCTCTACTGGCATTTATTTTaGGTGTGTTTTTTGGCAATATAT
CCTCACCAGCTTGTTCTGAAGACCATGAGGAGGTCATTTCTAATCA
AACATCAGTTATAGATTCTCAAAAAACAGAAATAGAAACTTTAAA
TAGCAAATTGTCTGATGCTGAACCATGGTTCAAAATGAAAGACGA
CGAAAAGAAAGCTATTGAAGCTGAAAATCAACGTAAAGCTGAAGA
AGCTAAAAAGGCTGAAGAACAACGTAAAAAAGAAGAAGAAGAGA
AGAAAGGATATGATACTGGTATTACTTATGACCAATTAGCTAGAA
CACCTGATGATTATAAGTACAAAAAGGTAAAATTTGAAGGTAAGG
TTATTCAAGTTATTGAAGATGGTGATGAGGTGCAAATAAGATTAG
CTGTGTCTGGAAATTATGATAAGGTCGTACTATGTAGTTATAAAAA
ATCAATAACTCCTTCAAGAGTGTTAGAGGATGATTACATAACTAT
AAGAGGTATAAGTGCTGGAACTATAACTTATGAATCAACTATGGG
TGGAAAAATAACCATACCAGGGATAGCTGTAGAGAAAGACGTCaaa
aaa

This construct has a LHA of 567 bp and a RHA of 555 bp, separated by the single base insertion as indicated above (ie. ‘a’).

The recombinant tcdC::117A allele was cloned as a ZraI fragment into the PmeI site of pMTL-SC7215 (FIG. 9), to give pMTL-SC7215::tcdC::117A. This vector was transferred into C. difficile R20291 by conjugation from E. coli CA434. Single and double cross-over clones were then isolated sequentially using the standardised protocol for codA mediated allele exchange as described above. Single and double cross-over clones were confirmed by PCR and sequencing (FIG. 15).

4) Use of codA Mediated Allele Exchange to Alter the Catalytic ‘DXD’ Domain of tcdB to ‘AXA’.

The recombinant tcdB allele (ie. tcdB-DXD286/8AXA) was constructed by DNA synthesis. This yielded the tcdB-DXD286/8AXA construct which had the following sequence (SEQ ID NO: 35):

GTTTAAACAAGATGTAAATAGTGATTATAATGTTAATGTTTTTTAT
GATAGTAATGCATTTTTGATAAACACATTGAAAAAAACTGTAGTA
GAATCAGCAATAAATGATACACTTGAATCATTTAGAGAAAACTTA
AATGACCCTAGATTTGACTATAATAAATTCTTCAGAAAACGTATG
GAAATAATTTATGATAAACAGAAAAATTTCATAAACTACTATAAA
GCTCAAAGAGAAGAAAATCCTGAACTTATAATTGATGATATTGTA
AAGACATATCTTTCAAATGAGTATTCAAAGGAGATAGATGAACTT
AATACCTATATTGAAGAATCCTTAAATAAAATTACACAGAATAGT
GGAAATGATGTTAGAAACTTTGAAGAATTTAAAAATGGAGAGTCA
TTCAACTTATATGAACAAGAGTTGGTAGAAAGGTGGAATTTAGCT
GCTGCTTCTGACATATTAAGAATATCTGCATTAAAAGAAATTGGTG
GTATGTATTTAGCTGTTGCTATGTTACCAGGAATACAACCAGACTT
ATTTGAGTCTATAGAGAAACCTAGTTCAGTAACAGTGGATTTTTGG
GAAATGACAAAGTTAGAAGCTATAATGAAATACAAAGAATATATA
CCAGAATATACCTCAGAACATTTTGACATGTTAGACGAAGAAGTT
CAAAGTAGTTTTGAATCTGTTCTAGCTTCTAAGTCAGATAAATCAG
AAATATTCTCATCACTTGGTGATATGGAGGCATCACCACTAGAAG
TTAAAATTGCATTTAATAGTAAGGGTATTATAAATCAAGGGCTAA
TTTCTGTGAAAGACTCATATTGTAGCAATTTAATAGTAAAACAAAT
CGAGAATAGATATAAAATATTGAATAATAGTTTAAATCCAGCTAT
TAGCGAGGATAATGATTTTAATACTACAACGAATACCTTTATTGAT
AGTATAATGGCTGAAGCTAATGCAGATAATGGTAGATTTATGATG
GAACTAGGAAAGTATTTAAGGTTTAAAC

This construct has a LHA of 509 bp and a RHA of 509 bp, separated by the DNA sequence CTGTTGC, where the bases boldface and underlined are altered from ‘A’ in the native chromosomal sequence, to ‘C’ in the recombinant sequence. This recombinant sequence encodes the amino acid sequence AXA in place of DXD in the active site of the toxin, thus rendering it completely non-toxic (Busch et al. (1998) 273:19566-19572. Journal of Biological Chemistry).

The recombinant tcdB-DXD286/8AXA allele was cloned into the PmeI site of pMTL-SC7315 (FIG. 2), to give pMTL-SC7315:: tcdB-DXD286/8AXA. This vector was transferred into C. difficile 630Δerm by conjugation from E. coli CA434. Single and double cross-over clones were isolated sequentially using the standardised protocol for codA mediated allele exchange as described above. Single and double cross-over clones were confirmed by PCR and sequencing (FIG. 16).

1. A method of double crossover homologous recombination in a host Clostridia cell comprising:
a first homologous recombination event between a donor DNA molecule and DNA of the host cell to form a product of the first recombination event in the host cell, wherein the donor DNA molecule comprises a codA gene and at least two homology arms; and
a second recombination event within the product of the first homologous recombination event, thereby to form a product of the second homologous recombination event in the host cell which is selectable by the loss of the codA gene.

2. The method of claim 1 wherein the DNA of the host cell comprises a chromosome or a plasmid of the host cell.

3. The method of claim 1 wherein the donor DNA molecule further comprises a selectable allele.

4. The method of claim 1 wherein the codA gene is a negative selection marker.

5. The method of any of claim 1 wherein the codA gene is a positive selection marker.

6. The method of claim 1 wherein the donor DNA molecule is not efficiently replicated in the host cell.

7. The method of claim 1 further comprising the step of selecting for products of the first homologous recombination event.

8. The method of claim 1 further comprising the step of selecting for products of the second homologous recombination event.

9. The method of claim 1 wherein the donor DNA molecule further comprises an alternative allele which is introduced into the host DNA in the first homologous recombination event.

10. The method of claim 9 wherein the alternative allele is retained in the host DNA following the second homologous recombination event.

11. The method of claim 1 wherein the donor DNA molecule comprises at least two homology arms.

12. The method of claim 1 wherein the donor DNA molecule is a plasmid.

13. The method of claim 12 wherein the plasmid is a non-replicative plasmid, a replication-defective plasmid or a conditional plasmid.

14. The method of claim 1 wherein the Clostridia cell is a species of the genus Clostridium or Thermoanaerobacterium saccharolyticum.

15. The method of claim 1 wherein the method further comprises the step of transforming a host Clostridia cell with a donor DNA molecule prior to the first homologous recombination event.

16. The method of claim 1 wherein the method further comprises the step of isolating the host cell comprising the product of the second homologous recombination event by virtue of the altered phenotype conferred by the loss of the codA gene.

17. The method of claim 1, wherein the donor DNA molecule comprises a polynucleotide sequence selected from any of the group comprising
SEQ ID NO: 2, SEQ ID NO: 3, SEQ ID NO: 4 and SEQ ID NO: 5.

18. A method of producing an altered host cell, comprising providing a host cell and carrying out the method of claim 1.

19. An altered host cell obtained by the method of claim 1.

20. A vector comprising the codA gene and at least two homology arms for the transformation of Clostridia cells.

21. A vector according to claim 20 comprising a sequence selected from any of SEQ ID NO: 2, SEQ ID NO: 3, SEQ ID NO: 4 and SEQ ID NO: 5.
